(19) United States
(12) Patent Application Publication
(10) Pub. No.: US 2014/0037618 A1
(43) Pub. Date: Feb. 6, 2014

(54) METHOD OF TREATING AUTOIMMUNE INFLAMMATORY DISORDERS USING IL-23R LOSS-OF-FUNCTION MUTANTS

(71) Applicant: GENENTECH, INC., SOUTH SAN FRANCISCO, CA (US)

(72) Inventors: Svetlana Pidasheva, San Francisco, CA (US); Hilary Clark, San Francisco, CA (US); Nico P. Ghilardi, Millbrae, CA (US); Timothy W. Behrens, Burlingame, CA (US); Hergen Spits, Amsterdam (NL); Sara Trifari, La Jolla, CA (US)

(21) Appl. No.: 13/902,714

(22) Filed: May 24, 2013

Related U.S. Application Data
(63) Continuation of application No. PCT/US2011/061892, filed on Nov. 22, 2011.
(60) Provisional application No. 61/417,113, filed on Nov. 24, 2010.

(51) Int. Cl. G01N 33/68; C12Q 1/68

(52) U.S. Cl. CPC G01N 33/6872; C12Q 1/6883
USPC 424/133.1; 435/6.11; 435/7.21; 435/7.24; 435/7.92

(57) ABSTRACT

The present invention relates to compositions and methods of diagnosing and treating autoimmune and inflammatory disorders that are characterized by IL-23R loss-of-function mutations.

### RELATED APPLICATION

This application is a continuation of International Application No. PCT/US2011/061892 having an international filing date of Nov. 22, 2011, which claims benefit under 35 U.S.C. §119 to U.S. Provisional Application No. 61/417,113 filed Nov. 24, 2010, the entire contents of each of which are incorporated herein in its entirety.

### SEQUENCE LISTING

This application contains a Sequence Listing submitted via EFS-Web and hereby incorporated by reference in its entirety. Said ASCII copy, created on Oct. 10, 2013, is named P4403R1C1SequenceLisitng.txt, and is 1,360 bytes in size.

### FIELD OF THE INVENTION

The present invention relates to the use of genetic polymorphisms in the diagnosis and treatment of autoimmune disorders and inflammatory disorders.

### BACKGROUND

Autoimmune inflammatory diseases (“AID”) are the manifestation or consequence of complex, often multiple interconnected biological pathways. In normal physiology, such interconnected pathways are critical to respond to insult or injury, initiate repair from insult or injury, and mount innate and acquired defense against foreign organisms. Disease or pathology occurs when these normal physiological pathways cause additional insult or injury, as a consequence of abnormal regulation or excessive stimulation, as a reaction to self, or any combination thereof.

The intervention at critical points in one or more of these pathways can have an ameliorative or therapeutic effect. Therapeutic intervention can occur by either antagonism of a detrimental process/pathway or stimulation of a beneficial process/pathway.

As used herein, the term inflammatory bowel disorder (“IBD”) is a subset of AID and describes a group of chronic relapsing and remitting inflammatory diseases of the gastrointestinal tract, of which the two primary subgroups are Chrohn's disease (CD) and ulcerative colitis (UC). The primary distinction between CD and UC are the location and nature of the inflammatory lesions. CD lesions can appear anywhere in the gastrointestinal tract, from mouth to anus, whereas in UC they appear only in the colon and rectum. While CD in adults typically manifests in the terminal ileum, in children the appearance is not so limited. While the inflammatory lesions of CD are discontinuous, transmural (affecting all layers of the bowel wall), and often granulomatous, in UC they are limited to the mucosal (epithelial lining of the gut). Patients with IBD are also much more likely to have other chronic inflammatory diseases, including primary sclerosing cholangitis, ankylosing spondylitis, psoriasis and multiple sclerosis. Urlep et al., Minerva Gastroenterol. Dietol. 51: 147-63 (2005).

Current evidence suggests that the inflammatory bowel diseases, Crohn's disease (CD) and ulcerative colitis (UC) are complex non-Mendelian polygenic disorders with important environmental interactions and stimuli. (Gaya et al. Lancet 2006; 367:1271-1284). The finding that variants of the NOD2/CARD15 gene are associated with susceptibility to CD is regarded as a landmark discovery and has catalysed widespread interest in the role of the innate and adaptive immune response in the development of CD. (Hugot et al. Nature 2001; 411:599-603; Ogura et al. Nature 2001; 411:603-606).

Polymorphisms in the IL-23R gene on chromosome 1p31 were observed to be associated with CD initially in a US study (Duerr et al. Science 2006; 314:1461-1463), and this has now been widely replicated in Europe catalyzing interest in the IL-23/Th17 pathway. (The Wellcome Trust Case Control Consortium, Nature 2007; 447:661-678). In the past 2 years, a number of genome-wide association studies (GWAS) in populations of European descent and a subsequent meta-analysis have identified 32 confirmed CD susceptibility genes/loci. (Barrett et al. Nat Genet. 2008, August; 40(8):955-62) These include innate immune genes that are specific to CD; NOD2, originally described in 2001 (Hugot et al. Nature 2001; 411(6837):599-603; Ogura et al. Nature 2001; 411(6837):603-6) and the autophagy genes ATG16L1 and IRGM (The Wellcome Trust Case Control Consortium, Nature 2007; 447:661-678), clearly indicating that defects in the intracellular processing of bacteria constitutes a central feature in the pathogenesis of CD. The discovery that germline variants of IL-23R were protective in CD coincided with murine experiments detailing the contribution of IL-23 (rather than IL-12 with which it shares the p40 subunit) to Th17 driven chronic intestinal inflammation. (Duerr et al. Science 2006; 314(5804):1461-3; Maloy et al. Mucosal Immunol 2008; 1(5):339-49). The meta-analysis and subsequent studies in UC have demonstrated that 3 other IL-23 signaling pathway genes (IL12B, JAK2 and STAT3) are all IBD susceptibility genes. (Barrett et al. Nat Genet. 2008, August; 40(8):955-62).

Several genes associated with Th17 inflammation have been linked with IBD susceptibility, including IL-23R, TNFSF15, STAT3, IL12B, CCR6 and JAK2. Barrett et al., Nat. Genet. 2008; 40: 955-62. Among these genes, the interleukin 23 receptor (IL-23R) gene (NM_{—}144701, GeneID: 149233) on chromosome 1p31 confers the highest odds ratio (“OD”) for disease development or lowest OD for disease protection. Duerr et al., Science 2006; 314: 1461-3; Yamazaki et al., Hum. Mol. Genet. 2005; 14: 3499-506; Rioux et al., Nat. Genet. 2007; 39: 596-604; Hampe et al., Nat. Genet. 2007; 39: 207-11; Libioulle et al., Plos Genet. 2007; 3:e58; Franke et al., PloS One 2007; 2:e691; Parkes et al., Nat. Genet. 2007; 39: 830-2; Raelson et al., Proc. Natl. Acad. Sci. USA 2007; 104:14747-52. IL-23R is also implicated in other chronic inflammatory diseases including psoriasis [Capon et al., Hum. Genet. 2007; 122: 201-6] and alkylosing spondylitis [Rueda et al., Ann. Rheum. Dis. 2008; 67: 1451-4]. The IL-23R^{R381Q} (rs11209026), a coding variant identified by GWAS, confers an OR of 0.45 for Crohn's disease development (MAF 1.9% in CD, 7% in controls, Duerr et al., supra), and an OR of 0.55 for UC development (MAF 3.7% in UC, 7% in controls, Silverberg et al., Nat. Genet. 2009; 41:216-20), implying a protective effect.

The IL-23R polymorphism Arg381Gln (R381Q), located in the cytoplasmic domain is known to be associated with a three-fold protective effect against developing CD. Duerr et al., Science 314: 1461-63 (2006); Abraham and Cho, Ann. Rev. Med., 60: 97-110 (2009). However, while the R381Q allele has been associated implicated stastistically to correlate with Crohn's, the biology or understanding of the protective effect is unkown.

Due to the hetereogenous nature of autoimmune and inflammatory inflammatory disorders, there is a great need to diagnose and treat particular patients in order to target the specific pathway disruption associated with the disease. While IL-23R has been associated with autoimmune and inflammatory disorders such as Crohn's disease, psoriasis and ankylosing spondylitis, and that the R381 coding variant for IL-23R has been found at lower frequencies in disease-affected individuals. Despite the fact that IL-23R antagonists have been proposed as therapeutics for the treatment of IBD, there is insufficient conclusive evidence of the actual involvement of IL-23 signaling in the disease. The discovery and confirmation herein that IL-23R signaling is not only pathogenic, but that patients having disrupted IL-23R can also develop disease, gives rise to the need for screening for IL-23R loss-of-function mutants in order to tailor a more suitable treatment regimen for their disorder, including excluding IL-23 pathway antagonists. Thus, there is a clear need for method of diagnosing or subtyping patients to identify patients suffering from autoimmune inflammatory disorders in which would not benefit from IL-23 pathway antagonists.

All publications mentioned herein are incorporated herein by reference to disclose and describe the information contained therein.

### SUMMARY

The present description of the invention provides for compositions and methods of diagnosing and treating autoimmune and inflammatory disorders. More specifically, such methods provide for diagnosing and/or treating autoimmune and inflammatory disorders (“AID”) that are characterized by IL-23R loss-of-function (“IL-23R LOF”) mutations.

In one embodiment, the description provides a method of advising a treatment regimen to a patient having at least one symptom of an AID comprising:

(a) analyzing a tissue sample from said patient for an IL-23R LOF mutation, and

(b) advising said patient or their care provider on treatment options based on the presence or
absence of said LOF mutation;

wherein (i) the presence of the IL-23R LOF mutation results in the administration of an agent other than an IL-23 pathway antagonist, and (ii) the absence of the IL-23R LOF mutation results in the administration of an agent that may include an IL-23 pathway antagonist.

In another embodiment, the description provides a method of treating a patient having at least one symptom of an AID comprising:
(a) analyzing a tissue sample from said patient for an IL-23R LOF mutation, and
(b) administering at least a therapeutically effective amount of a therapeutic based on the presence or absence of said IL-23R LOF mutation;

wherein the therapeutic administered, includes (i) an agent other than an IL-23 pathway antagonist when an IL-23R LOF mutation is present, and (ii) an agent that may include an IL-23 pathway antagonist if an IL-23R LOF mutation is not present. In one specific aspect, the method provides for administering an IL-23 pathway antagonist when an IL-23R LOF mutation is not found in the tissue sample. In another specific aspect, the patient has an AID. In yet another aspect, the mutation results in the polymorphism R381Q. In yet a further specific aspect, the polymorphism results from the SNP rs11209026. In yet a further aspect the polymorphism results from a SNP selected from the group consisting of: rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, rs11209003. In yet a further aspect, the other than IL-23 pathway antagonist is one or more agents selected from the group consisting of: an aminosalicylate, a corticosteroid, an immunosuppressive agent, an antibody targeting other than a component of the IL-23 pathway or antigen-binding fragment of such antibody, an antibiotic and anti-metabolic agent and a palliative therapy. In yet a further aspect, the AID is selected from the group consisting of: ankylosing spondylitis, inflammatory bowel disease (“IBD”), dermatomyositis and rheumatoid arthritis. In yet a further aspect, the AID is IBD. In yet a further specific aspect, IL-23 pathway antagonist is directed against one or more IL-23 pathway components selected from the group consisting of: p40 (IL-12B), p19 (IL-23A), IL-12RB1, IL-23R, TYK2, JAK2, STAT-3.

In yet another embodiment, the description provides a method for treating a patient having at least one symptom of chronic inflammation comprising:
(a) analyzing a tissue sample from said patient for an IL-23R LOF mutation, and
(b) administering at least a therapeutically effective amount of a therapeutic based on the presence or absence of said LOF mutation;
   wherein the therapeutic administered, includes (i) an agent other than an IL-23 pathway antagonist when an IL-23R LOF mutation is present, and (ii) an agent that may include an IL-23 pathway antagonist if an IL-23R LOF mutation is not present. In one aspect, the method provides for administering an IL-23 pathway antagonist when an IL-23R LOF mutation is not found in the tissue sample. In another aspect, the patient has an AID. In yet another aspect, the mutation results in the polymorphism R381Q. In yet a further specific aspect, the polymorphism results from the SNP rs11209026. In yet a further aspect, the polymorphism results from the SNP selected from the group consisting of: rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, rs11209003. In yet a further specific aspect, the other than IL-23 pathway antagonist is one or more agents selected from the group consisting of: an aminosalicylate, a corticosteroid, an immunosuppressive agent, an antibody or antibody derivative, an antibiotic and anti-metabolic agent and a palliative therapy. In yet a further specific aspect, the IL-23 pathway antagonist is directed against one or more IL-23 pathway components selected from the group consisting of: p40 (IL-12B), p19 (IL-23A), IL-12RB1, IL-23R, TYK2, JAK2, STAT-3. In yet a further specific aspect the AID is selected from the group consisting of: ankylosing spondylitis, inflammatory bowel disease (“IBD”), dermatomyositis and rheumatoid arthritis. In yet a further aspect, the AID is IBD. In yet a further aspect, the tissue sample is derived from a colonic tissue biopsy. In a preferred embodiment, the biopsy is a tissue selected from the group consisting of terminal ileum, the ascending colon, the descending colon, and the sigmoid colon. In yet further aspects, the biopsy is from an inflamed colonic area or from a non-inflamed colonic area. The inflamed colonic area may be acutely inflamed or chronically inflamed.

In a further embodiment, the description provides for a method for assessing the function of an IL-23 responsive cell, comprising: (a) isolating an IL-23 responsive cell, (b) detecting an IL-23R LOF mutant in said cell, and (b) wherein the presence of IL-23R LOF mutant correlates to diminished cell function. In one aspect, the diminished function is Th17-induced inflammation. In another aspect, the diminished function is surface expression of IL-23R. In yet another aspect, the diminished function is a reduced Th17 cytokine response profile. In yet another aspect, the diminished function is reduced STAT3 phosphorylation. In yet another aspect, the diminished function is reduced expression of the transcription factor RORγt. In yet another aspect, the diminished function is reduced expression of GATA-3. In yet another aspect, the diminished function is reduced expression of MMP9. In yet a further aspect, the IL-23 responsive cell is selected from the group consisting of: dendritic cells, T cells, including αβ and γδ T cells, NK cells, including NKL, monocytes, macrophages, B cells αβ and γδ cells as well as innate leukocytes. In yet a further specific aspect, the IL-23 responsive cell is a T cell.

In a further embodiment, the description provides a composition for treating an AID, comprising a detective agent for detecting an IL-23R LOF mutation, wherein (i) the presence of the IL-23R LOF mutation in a tissue sample results in the administration of an agent other than an IL-23 pathway antagonist, and (ii) the absence of an IL-23R LOF mutation may result in the administration of an IL-23 pathway antagonist. In a specific aspect, the IL-23R LOF mutation results in the IL-23R polymorphism R381Q. In another specific aspect, the R381Q polymorphism results from the SNP rs11209026. In yet a further aspect, the polymorphism results from the SNP selected from the group consisting of: rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, rs11209003.

In a further embodiment, the description provides for the use of an IL-23R LOF mutation in the manufacture of a medicament for the treatment of an AID, wherein (i) the presence of an IL-23R LOF mutation in a tissue sample results in the administration of an agent other than an IL-23 pathway antagonist, and (ii) the absence of an IL-23R LOF mutation may result in the administration of an IL-23 pathway antagonist. In a specific aspect, the IL-23R LOF mutation results in the IL-23R polymorphism R381Q. In another specific aspect, the R381Q polymorphism results from the SNP rs11209026. In yet a further aspect, the polymorphism results from the SNP selected from the group consisting of: rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, rs11209003.

For all aspects, the method may further comprise the step of creating a report summarizing the results of the described method.

For all aspects, the method of detecting an IL-23R LOF mutation may includes one or more of the following: (i) northern blotting, (ii) in situ hybridization, (iii) RNAse protection assays, (iv) reverse transcription polymerase chain reaction (RT-PCR), (v) anti-nucleic acid antibodies may be employed that can recognize specific duplexes, including (a) DNA duplexes, (b) RNA duplexes, (c) DNA-RNA hybrid duplexes or (d) DNA-protein duplexes, (vi) gene expression profiling, (vii) polymerase chain reaction (PCR) including quantitative real time PCR (qRT-PCR), (viii) microarray analysis, such as by using the Affymetrix® GenChip technology, (ix) serial analysis of gene expression (SAGE), (x) MassARRAY, Gene Expression Analysis by Massively Parallel Signature Sequencing (MPSS), (xi) proteomics, (xii) immunohistochemistry (IHC), (xiii) gene specific priming, (xiv) promoter methylation analysis, (xv) intron based probes/primers.

These and further embodiments and aspects will be apparent to those of ordinary skill in the art.

### BRIEF DESCRIPTION OF DRAWINGS

FIGS. 1A-G show that the arginine at position 381 of the IL-23R is absolutely conserved across different species. FIG. 1A is a gene map of exons encoding the IL-23R gene.

FIG. 1B is a sequence alignment of the IL-23R protein sequences from different species. FIG. 1C is a flow cytometric analaysis of BaF3 cell clones retrovirally transduced with IL23R^{Q381} and IL23R^{R381}. The clones were selected to express equal levels of extracellular IL-23R (1 out of 2 pairs of clones shown here) selected by FACS. FIG. 1D shows a geometric MFI of IL23R corresponding to the chart shown in FIG. 1C. FIG. 1E is a bar graph of the real-time PCR analysis for IL-23R and IL12RB1 expression, presented (in arbitrary units (AU) relative to the expression of the “housekeeping gene” RPL19. mRNA was collected from BaF3 cells transfected with IL-23R^{R381} and IL-23R^{Q381} clones. FIGS. 1F (plot) and 1G (MFI graph) show through flow cytometric analysis that IL-23R^{Q381} clones stimulated with sub maximal levels of IL-23 had decreased STAT3 phosphorylation compared to the IL-23R^{R381}. Isotype controls of non-stimulated samples are indicated by light gray shading, IL-23R^{R381} by a grey line and IL-23R^{Q381} by a black line. Data are representative of at least three independent experiments.

FIGS. 2A-E show a flow cytometric analyses of T cell lines from IL-23R^{R381} and IL-23R^{Q381} donors. The numbers in the quadrants indicate the percent cells. Untransformed polyclonal IL-23R^{Q381} positive T cells have decreased IL-23R surface expression and reduced IL-23 responsiveness compared to IL-23^{R381} T cell lines. FIG. 2A shows that IL-23R cell surface expression on non-permeabilized T cells of representative donors, and FIG. 2B shows the percent mean (n=4), and standard error. FIG. 2C shows the IL-23 and IL-6 induced pSTAT3 response in representative donors, while FIG. 2D reports the mean and standard error of 4 donors. FIG. 2E shows the MFI of pSTAT3 positive population (n=4). The data are representative examples of at least three independent experiments.

FIGS. 3A-C show that untransformed polyclonal IL-23R^{Q381} positive T cells have decreased IL-23 induced pSTAT1 and pSTAT5 compared to IL-23R^{R381}T cell lines. FIG. 3A shows that IL-23 and IL-2 induced pSTAT5, while FIG. 3B shows that IL-23 induced pSTAT1 response in representative donors. FIG. 3C shows the mean percent of the persent positive and MFI of pSTAT1 and pSTAT5 (n=4). The data are representative examples of at least two independent experiments.

FIGS. 4A-C show a flow cytometric analysis of PBMCs from IL-23R^{R381} compared to IL23R^{Q381}, showing that IL-23R^{Q381} positive donors have similar numbers of Th17 cells compared to IL-23R^{R381} donors. The numbers in the quadrants indicate percentage cells.

FIG. 4A shows gating strategies for CD45RA⁻CCR6⁺CCR4⁺CXCR3⁻ cells of representative donors on non-permeabilized PBMC (CD4-enriched). FIG. 4B shows gating strategies for CD4⁺CD45RO⁺CD161⁺IL1R1⁺ cells of representative donors on non-permeabilized PBMC. FIG. 4C shows the percent mean (n=3), and error bars.

FIGS. 5A-F report the ICS of cytokine production by T cell lines stimulated with anti-CD3/CD28 dynabeads, and shows that peripheral blood cytokine levels are comparable in IL-23R^{Q381} and IL-23R^{R381} positive donors. FIG. 5A shows representative donors for IL-22 and IL-17A, FIG. 5B shows IL-10 and IFN-γ levels. FIG. 5C shows the frequency of cyotokine positive (i.e., IL-17A and IL-22) cells (n=4). FIG. 5D shows the cytokine production by PBMCs stimulated with anti-CD3 and anti-CD28 antibodies with (+) and without (−) IL-23, as measured by ELISA (n=3). FIG. 5E shows serum IL-22 levels in IL-23R^{Q381} and IL-23R^{R381} positive donors (n=5), as measured by ELISA. FIG. 5F shows real-time PCR analysis for IL-23R and RORC mRNA expression, presented in arbitrary units (AU) relative to the expression of the “housekeeping gene” GAPDH. Each symbol represents an individual donor. Data are representative examples of at least two independent experiments.

FIG. 6 is summary of the results obtained in this study using genotype-selectable normal donors. IL-23RQ381 donors had significantly reduced IL-23R surface expression on T cells, leading to the decreased IL-23 induced STAT3 phosphorylation. Decreased STAT3 signaling in T cells like Th17 might modulate the extent and duration of the response in the host, leading to decreased secretion of proinflammatory cytokines such as IL-17 and IL-22 in the gut. The lowered expression levels explain the protective effective of R381Q variants in CD and other autoimmune disorders.

FIGS. 7A-D show a flow cytomatric analyses from IL-23R^{R381} and IL-23R^{Q381} donors showing that PBMCs from IL-23R^{Q381} positive donors have decreased IL-23R surface expression and reduced IL-23 responsiveness compared to IL-23R^{R381} donors. The numbers in each quadrant indicate percent cells. FIG. 7A shows IL-23R cell surface expression on non-permeabilized PBMC stimulated with anti-CD3 and anti-CD28 antibodies for 72 hours of representative donors. FIG. 7B shows the percent mean of 4 donors per group. The error bars indicate the standard error of the mean. FIG. 7C shows that IL-23 and IL-6 induced pSTAT3 response in whole blood in representative donors, with 4 donors per group (FIG. 7D). The data are representative examples of at least three independent experiments.

FIGS. 8A-C show an ICS report of cytokine production by PBMCs stimulated with anti-CD3/CD28 dynabeads, showing that cytokine levels are comparable in IL-23R^{Q381} and IL-23R^{R381} positive donors. FIG. 8A shows representative donors for IL-22 and IL-17, while FIG. 8B shows IL-10 and IFN-γ. FIG. 8C represents the data in bar graph form (n=4). The data are representative examples of at least three independent experiments.

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “IL-23 signaling pathway” or “IL-23 pathway” includes all components that provide and/or receive a signal resulting from the binding of the cytokine IL-23, with its receptor, including the resulting intracellular signal transduction and resulting in the nuclear transcription activation. For example, this includes the IL-23 ligand components p40 (IL-12B) and p19 (IL-23A), and the receptor components IL-12RB1 and IL-23R, the receptor binding components TYK2 and JAK2, and the cytoplasmic signal transducer STAT-3. Additionally, cyokine production resulting from IL-23 signaling, “downstream IL-23 cytokines,” includes IL-1, IL-6, IL-17A, IL-17F, IL-21, IL-22, IL-26 and TNF-α and GM-CSF, chemokines (e.g., KC, MCP-1, MIP-2) and matrix metalloproteases.

The term “IL-23 pathway antagonist” includes any therapeutic agent that blocks, attenuates, or reduces the activity of one or more components of the IL-23 signaling pathway so as to reduce or arrest IL-23 signaling pathway transduction, the nuclear transcription activation, and/or the enhanced downstream cytokine production resulting therefrom. Specific examples includes antagonists that prevent the interaction of IL-23 ligand with IL-23 receptor [e.g., anti-ligand, including components p40 (IL-12B) and p19 (IL-23A) antagonists, anti-receptor, including components IL-12RB1 and IL-23R antagonists], antagonists which prevent the interaction of the receptor binding components (e.g., TYK2, JAK2), and intracellular signaling molecules (e.g., STAT-1, STAT-3, STAT-4, STAT-5). The form of such IL-23 pathway antagonists can include antibodies, and antigen binding fragments thereof, small molecules, interfering RNA (“RNAi”, e.g.—siRNA, shRNA, miRNA), oligopeptides, etc. However, the term “IL-23 pathway antagonist,” as intended for use herein, does not apply to antagonists that block the activity of downstream cytokines associated with IL-23 pathway activation (except that which directly results from such activation) resulting in activation of Th-17 cells (e.g., IL-17A, IL-17F, IL-21, IL-22, IL-26 and TNF-α). Said another way, the scope of the term “IL-23 pathway antagonist” extends only to the component of Th-17 inflammation that is directly attributable to IL-23 pathway activation. For example, while the activity of an IL-23 pathway antagonists may include a measurement of decreased expression of one or more such downstream cytokines, it is not measured by the activity of such downstream cytokine itself.

A “small molecule” or “small organic molecule” is one that has a molecular weight below about 500 Daltons.

An “interfering RNA” “RNAi” is RNA of 10 to 50 nucleotides in length which reduces expression of a target gene, wherein portions of the strand are sufficiently complementary (e.g., having at least 80% identity to the target gene). The method of RNA interference refers to the target-specific suppression of gene expression (i.e., “gene silencing”), occurring at a post-transcriptional level (e.g., translation), and includes all post-transcriptional and transcriptional mechanisms of RNA mediated inhibition of gene expression, such as those described in P. D. Zamore, Science 296: 1265 (2002) and Hannan and Rossi, Nature 431: 371-378 (2004). As used herein, RNAi can be in the form of small interfering RNA (siRNA), short hairpin RNA (shRNA), and/or micro RNA (miRNA). Such RNAi molecules are often a double stranded RNA complexes that may be expressed in the form of separate complementary or partially complementary RNA strands. Methods are well known in the art for designing double-stranded RNA complexes. For example, the design and synthesis of suitable shRNA and siRNA may be found in Sandy et al., BioTechniques 39: 215-224 (2005). RNAi may be identified and synthesized using known methods (Shi Y., Trends in Genetics 19(1):9-12 (2003), WO/2003056012 and WO2003064621), and siRNA libraries are commercially available, for example from Dharmacon, Lafayette, Colo.

A “small interfering RNA” or siRNA is a double stranded RNA (dsRNA) duplex of 10 to 50 nucleotides in length which reduces expression of a target gene, wherein portions of the first strand is sufficiently complementary (e.g., having at least 80% identity to the target gene). siRNAs are designed specifically to avoid the anti-viral response characterized by elevated interferon synthesis, nonspecific protein synthesis inhibition and RNA degredation that often results in suicide or death of the cell associated with the use of RNAi in mammalian cells. Paddison et al., Proc Natl Acad Sci USA 99(3): 1443-8. (2002).

The term “hairpin” refers to a looping RNA structure of 7-20 nucleotides. A “short hairpin RNA” or shRNA is a single stranded RNA 10 to 50 nucleotides in length characertized by a hairpin turn which reduces expression of a target gene, wherein portions of the RNA strand are sufficiently complementary (e.g., having at least 80% identity to the target gene). The term “stem-loop” refers to a pairing between two regions of the same molecule base-pair to form a double helix that ends in a short unpaired loop, giving a lollipop-shaped structure.

A “micro RNA” or “miRNA” (previously known as stRNA) is a single stranded RNA of about 10 to 70 nucleotides in length that are initially transcribed as pre-miRNA characterized by a “stem-loop” structure, which are subsequently processed into mature miRNA after further processing through the RNA-induced silencing complex (RISC).

An “RNAi” suitable for use with the present invention binds, preferably specifically, to a nucleic acid encoding an IL-23 signaling pathway component, and reduces its expression. This means the expression of such IL-23 signaling pathway component molecule is lower with the RNAi present as compared to expression of the molecule in a control where such RNAi is not present. Suitable RNAi may be identified and synthesized using known methods (Shi Y., Trends in Genetics 19(1): 9-12 (2003), WO2003056012, WO2003064621, WO2001/075164, WO2002/044321.

Oligopeptides suitable for use in the present invention bind, preferably specifically, to an IL-23 signaling pathway component, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

Small molecule antagonists suitable for use in the present invention are organic molecules other than an oligopeptide or antibody, as defined herein, that inhibits, preferably specifically, an IL-23 pathway component. Example small molecules may be identified and chemically synthesized using known methodology (see, e.g., PCT Publication Nos. WO2000/00823 and WO2000/39585). Such BNCA small molecules are usually less than about 2000 daltons in size, alternatively less than about 1500, 750, 500, 250 or 200 daltons in size, are capable of binding, preferably specifically, to a B7 negative stimulatory polypeptide as described herein, and may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

The term “IL-23R loss-of-function” (“IL-23R LOF”) mutant means a mutation in the gene allele encoding a component of the IL-23 receptor signaling pathway that results in reduced or abolished IL-23 signaling activity. Such mutations can inhibit the interaction of IL-23 signaling pathway components, or they can impair the positions or placement of IL-23R pathway components, or they can otherwise impair the contribution of the IL-23R signaling pathway component to IL-23 signal transduction relative to wild type. Examples of reduced (relative to wild-type) IL-23 signaling activity are any one or more of the following: (i) reduced cell surface expression of IL-23R and/or decreased number of IL-23R positive/responsive cells, (ii) decreased STAT-1, 3, 4, or 5 phosphorylation upon IL-23 stimulation, (iii) decreased expression/secretion of downstream cytokines such as IL-17 and IL-22. Example IL-23R loss-of-function mutants include: the R381Q polymorphism in IL-23R(RS11209026). In addition, the following IL-23R SNPs are associated with IBD: rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008 and rs11209003.

Inflammation, broadly defined, is an immune system response in which white blood cells migrate out of blood vessels to act as phagocytes on a target molecule. Inflammation progresses through the four stages of (1) redness, (2) heat, (3) swelling, and (4) pain. Additional symptoms can include flu-like symptoms of fever, chills, fatigue, loss of energy and headaches. Two common forms of inflammation include acute—which is of short duration and typically occurs in response to infection, and chronic—which is of long duration and can be brought on by the acute form or an AID. Chronic information may also have a slow onset, and the symptoms may not be as server as in acute form. Chronic inflammation is also mediated by macrophages and lymphocytes. A key symptom of chronic inflammation is collagen production, which can lead to fibrosis—resulting in scarring and permanent distortion of the affected tissue. Additionally, useful markers of chonic inflammation include elevated levels of: (1) C-reactive protein (CRP), (2) fibrinogen, and (3) IL-6.

As a result, a “symptom of chronic inflammation” can be (i) any symptom of inflammation (e.g., one or more flu-like symptoms of fever, chills, fatigue, loss of energy, headache), (ii) elevated levels of macrophages and lymphocytes, (iii) collagen production, (iv) fibrosis and (v) elevated expression of one or more of the following: (1) C-relative protein, (2) fibrogen or (3) IL-6.

“Th17-induced inflammation” or “Th17 inflammation” is inflammation resulting from activation of Th17 CD4⁺ T-cells, as opposed to inflammation that results from Th1 or Th2 cell activation. Th-17 inflammation has been strongly implicated in autoimmune conditions. While IL-6 and TGF-β combined can induce differentiation of Th-17 cells from naïve T-cells, IL-23 can induce proliferation of Th-17 cells from memory T-cells. Cytokines other than IL-23 may also be important to maintain a Th-17 cell inflammatory response (e.g., IL-1 etc.), while the attenuation of others (e.g., IL-2, IL-4, IFN-α, IFN-γ, etc.) augments or sustains it. A transcription factor that is both distinctive and necessary of Th-17 inflammation is RORγt. Cytokines associated with Th17 inflammation (“Th-17 cytokine response profile”), and hence the presence and/or hyperactivity of which may be used to indicated that Th17 inflammation is present include IL-17A, IL-17F, IL-21, IL-22, IL-26 and TNF-α.

The term “reduced Th-17-induced inflammation” is a significant and measurable reduction in one or more aspects of Th17-induced inflammation as previously described. Examples of reduced Th-17 induced inflammation include a significant and measurable reduction (relative to physiologically normal response) in one or more of: (1) surface expression of IL-23R, (ii) Th17 cytokine response profile, (iii) STAT3 phosphorylation, (iv) expression of the transcription factor RORyt.

The term “autoimmune inflammatory disorder” or “AID” includes disorders or diseases that are inflammatory (i.e., symptomatic of inflammation) but also autoimmmune (i.e., host immune system attacks self antigens) in nature, manifesting clinically with the symptoms of chronic inflammation, and result in the simultaneous destruction and healing of body tissues. AID disorders are mediated primarily by mononuclear cells (e.g., monocytes, macrophages, lymphocytes, plasma cells) and fibroblasts, and the secreted fators: IFN-γ, inflammatory cytokines, growth factors, reactive oxygen species and hydrolytic enzymes. Specific examples of AID include: ankylosing spondylitis, inflammatory bowel disease (e.g., Crohns Disease, ulcerative colitis), dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, lupus erythematosus, mixed Connective Tissue Disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, sarcoidosis, schizophrenia, scleroderma, Sjögren's syndrome, stiff person syndrome, yemporal arteritis (also known as “giant cell arteritis”), uveoretinitis, vasculitis, vitiligo, Wegener's granulomatosis. In a more specific aspect, AID includes ankylosing spondylitis, inflammatory bowel disease, dermatomyositis, rheumatoid arthritis.

The term “other than IL-23 pathway antagonist” includes one or more therapeutic that is not an IL-23 pathway antagonist, but is useful or known to be useful to treat AID. For example, in the treatment of the AID disorder IBD, various therapeutics that are not IL-23 pathway antagonists may be perscribed. Examples drugs are: (1) Aminosalicylates/anti-inflammatory drugs-sulfasalazine (Azulfidine®), mesalamine (Asacol®, Rowasa®, Pentasa®), olsalazine (Depentum®) and balsalazide (Colazal®); (2) Corticosteroids—methylprednisolone, hydrocortisone, prednisone, prednisolone, budesonide, dexamethasone; (3) Immune system suppressors—azathioprine (Imuran®) and mercaptopurine (Purinethol®). (4) Antibodies, and antibody derivatives (biologics)—(i) anti-TNF-α antibody: infliximab/Remicade®, adalimumab/Humira®, certolizumab pegol/Cimzia®, and (ii) serum immunoglobulins—Sandimmune®, natalizumab/Tysabri®; (5) Antibiotics—metronidazole/Flagyl®, ciprofloxacin/Cipro®, Neoral®; (6) Anti-metabolic agents—methotrexate/Rheumatrex®; (7) Palliative therapies—anti-diarrheals, laxatives, pain relievers, iron supplements, nutrition, vitamin B-12, calcium and vitamin D, TNF antagonist (non-biologic, thalidomide), cyclosporine A, nicotine patch, butyrate enema, heparin.

The term “IL-23 responsive cell” includes any cell that is regulated, modulated or affected, including survival, by IL-23 signaling. Such cells include dendritic cells, T cells, NK cells (including NKL), monocytes, macrophages, B cells (Parham et al., J. Immunol. 168: 5699-5708 (2002)), αβ and γδ T cells as well as innate leukocytes (Awasthi et al., J. Immunol. 182 (2009), pp. 5904-5908).

The term “inflammatory bowel disease” or “IBD” is used as a collective term for ulcerative colitis and Crohn's disease. Although the two diseases are generally considered as two different entities, their common characteristics, such as patchy necrosis of the surface epithelium, focal accumulations of leukocytes adjacent to glandular crypts, and an increased number of intraepithelial lymphocytes (IEL) and certain macrophage subsets, justify their treatment as a single disease group.

Although the precise etiology of IBD is unkown, epidemiological data conclusively points to a dysregulation of the immune response againsts the luminal flora in a genetically susceptible host. While the initial immune dysregulation may be prompted by an acute infective trigger [Rodriguez et al., Gastroenterology 130: 1588-94 (2006); Porter et al., Gastroenterology 135: 781-86 (2008)], the event is not likely resulting from traditional pathogens. However, the alternation of the composition and function of the microbiome (as either a primary or a secondary phenomenon) is the subject of intense investigation. While other environmental factors are thought to be involved, genetic epidemiologiacal observations in CD v. UC has focued an increased emphasis and study on the genetic components of CD.

A diagnosis of IBD is generally by colonoscopy with biopsy of pathological lesions. In any event, an examination of morphology alone can be inconclusive and result in a diagnosis of “indeterminate colitis.” While there are serum antibody tests that can help in the diagnosis, they are not necessarily conclusive. The antibodies are “perinuclear anti-neutrophil antibody” (pANCA) and “anti-Saccharomyces cervisiae antibody” (ASCA). Most patients with UC have the pANCA antibody, but not the ASCA, antibody, while most patients with Crohn's disease have the ASCA antibody but not the pANCA one.

The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions, and the development of fistulas and fissures are not uncommon. As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine).

The term “ulcerative colitis” or “UC” is used herein to refer to a condition involving inflammation of the large intestine and rectum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon.

The term “inactive” AID is used herein to mean an AID that was previously diagnosed in an individual but is currently in remission. This is in contrast to an “active” AID in which an individual has been diagnosed with and AID but has not undergone treatment. In addition, the active AID may be a recurrence of a previously diagnosed and treated AID that had gone into remission (i.e. become an inactive AID). Such recurrences may also be referred to herein as “flare-ups” of an AID. Mammalian subjects having an active autoimmune disease, such as an IBD, may be subject to a flare-up, which is a period of heightened disease activity or a return of corresponding symptoms. Flare-ups may occur in response to severe infection, allegic reactions, physical stress, emotional trauma, surgery, or environmental factors.

The term “modulate” is used herein to mean that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator.

The terms “antagonize,” “inhibit”, “down-regulate”, “underexpress” and “reduce” are used interchangeably and mean that the expression of a gene, or level of RNA molecules or equivalent RNA molecules encoding one or more proteins or protein subunits, or activity of one or more proteins or protein subunits, is reduced relative to one or more controls, such as, for example, one or more positive and/or negative controls. An “antagonist” is an agent that exhibits one or more of these properties.

The term “up-regulate” or “overexpress” is used to mean that the expression of a gene, or level of RNA molecules or equivalent RNA molecules encoding one or more proteins or protein subunits, or activity of one or more proteins or protein subunits, is elevated relative to one or more controls, such as, for example, one or more positive and/or negative controls.

The term “diagnosis” is used herein to refer to the identification of a molecular or pathological state, disease or condition, such as the identification of AID.

The term “prognosis” is used herein to refer to the prediction of the likelihood of AID development or progression, including autoimmune flare-ups and recurrences following surgery. Prognostic factors are those variables related to the natural history of AID, which influence the recurrence rates and outcome of patients once they have developed AID. Clinical parameters that may be associated with a worse prognosis include, for example, an abdominal mass or tenderness, skin rash, swollen joints, mouth ulcers, and borborygmus (gurgling or splashing sound over the intestine). Prognostic factors may be used to categorize patients into subgroups with different baseline recurrence risks.

The “pathology” of an AID includes all phenomena that compromise the well-being of the patient. In IBD, pathology is primarily attributed to abnormal activation of the immune system in the intestines that can lead to chronic or acute inflammation in the absence of any known foreign antigen, and subsequent ulceration. Clinically, IBD is characterized by diverse manifestations often resulting in a chronic, unpredictable course. Bloody diarrhea and abdominal pain are often accompanied by fever and weight loss. Anemia is not uncommon, as is severe fatigue. Joint manifestations ranging from arthralgia to acute arthritis as well as abnormalities in liver function are commonly associated with IBD. During acute “attacks” of IBD, work and other normal activity are usually impossible, and often a patient is hospitalized.

The term “treatment” refers to both therapeutic treatment and prophylactic or preventative measures for AID, wherein the object is to prevent or slow down (lessen) the targeted pathologic condition or disorder. Those in need of treatment include those already with an AID as well as those prone to have an AID or those in whom the AID is to be prevented. Once the diagnosis of an AID has been made by the methods disclosed herein, the goals of therapy are to induce and maintain a remission.

The term “test sample” refers to a sample from a mammalian subject suspected of having an AID, known to have an AID, or known to be in remission from an AID. The test sample may originate from various sources in the mammalian subject including, without limitation, blood, saliva, skin, semen, serum, urine, feces, bone marrow, mucosa, tissue, etc., including a tissue biopsy of the gastrointestinal tract including, without limitation, ascending colon tissue, descending colon tissue, sigmoid colon tissue, ileocolon, and terminal ileum tissue.

The term “control” or “control sample” refers a negative control in which a negative result is expected to help correlate a positive result in the test sample. Controls that are suitable for the present invention include, without limitation, a sample known to have normal levels of gene expression, a sample obtained from a mammalian subject known not to have an AID, and a sample obtained from a mammalian subject known to be normal. A control may also be a sample obtained from a subject previously diagnosed and treated for an AID who is currently in remission; and such a control is useful in determining any recurrence of an AID in a subject who is in remission. In addition, the control may be a sample containing normal cells that have the same origin as cells contained in the test sample. Those of skill in the art will appreciate other controls suitable for use in the present invention.

The term “microarray” refers to an ordered arrangement of hybridizable array elements, preferably polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate), 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The terms “splicing” and “RNA splicing” are used interchangeably and refer to RNA processing that removes introns and joins exons to produce mature mRNA with continuous coding sequence that moves into the cytoplasm of an eukaryotic cell.

The term “exon” refers to any segment of an interrupted gene that is represented in the mature RNA product (B. Lewin. Genes IV Cell Press, Cambridge Mass. 1990). The term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and having GT and AG splice consensus sequences at their 5′ and 3′ boundaries.

A “native sequence” polypeptide is one which has the same amino acid sequence as a polypeptide derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

In the context of antibody domains, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30- | (Kabat |
| | | | | H35B | Numbering) |
| H1 | H31-H35 | H26-H35 | H26-H32 | H30-H35 | (Chothia |
| | | | | | Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pliickthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a small molecule or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g. deamidated antibody variant), a basic variant, an antibody with an amino-terminal leader extension (e.g. VHS-) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

### B. General Description of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

1. IL-23 Signaling in Inflammation

The importance of genetic associations in the interleukin 12 (IL-23) pathway in multiple autoimmune inflammatory disorders, including inflammatory bowel disease (IBD), has conincided with significant advances in the understanding of its key role in host defense and organ-specific autoimmunity. In particular, genetic polypmorphisms in the IL-23 receptor, IL-23R, represent one of the strongest associations in Crohn's disease (CD) and are also associated in ulcerative colitis (UC) [Duerr et al., Science 314: 1461-63 (2006)], psoriasis [Cargill et al., Am. J. Hum. Genet. 80: 273-90 (2007)], and alkylosing spondylitis [Burton et al., Nat. Genet. 39: 1329-37 (2007)].

The presence of multiple SNPs in the IL-23R gene region having distinct correlation patterns to CD is suggestive of the presence of multiple independent risk and susceptibility alleles. Duerr et al., supra. A notable SNP demonstrates association with the polymorphism Arg381Gln is located in the cytoplasmic domain of the receptor. Duerr et al., supra. This less common glutamine allele confers an approximately 3-fold protection against not only developing CD, but also other diseases such as psoriasis [Capon et al., Hum. Genet. 122: 201-206 (2007)] and ankylosing spondylitis. [Rudea et al., Ann. Rheum. Dis. 67: 1451-1454 (2008)]. This indicates a protective role in (or at least an association with a lower risk of developing) these diseases. In addition, IL-23 pathway genes have been shown to be associated with CD, indicating the importance of the proper regulation of this pathway in maintaining intestinal immune homeostasis and suggesting a key role for IL-23R in the disease pathogenesis in multiple populations.

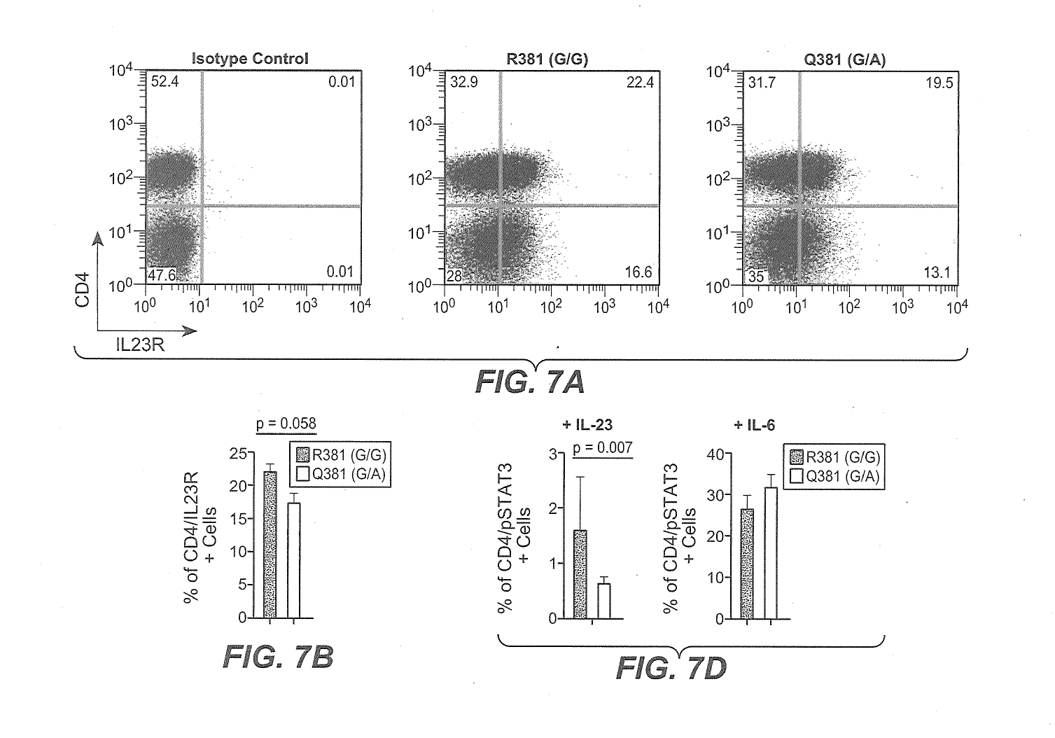

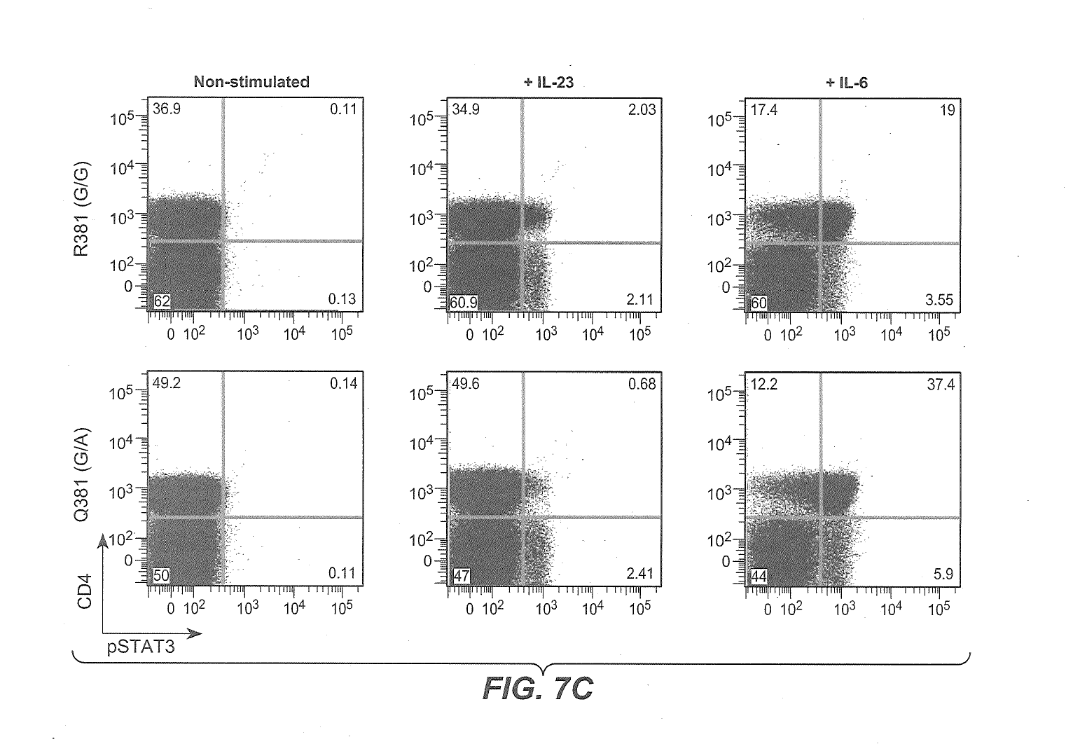

IL-23 was cloned in 2002 by Parham et al. Parham et al., J. Immunol. 168: 5699-5708 (2002). It is mainly expressed on activated T cells, NK cells and at low levels on monocytes, macrophages and dendritic cells, as a heterodimer consisting of a unique IL-23Ra chain and an IL-12R1 chain, a subunit also shared with IL-12R. Upon activation by IL-23 cytokine, which is composed of a unique p19 subunit and a shared p40 subunit with IL-12, it signals through the JAK/STAT pathway. Oppman et al., Immunity 13: 715-725 (2000). IL-23R associates constitutively with JAK2, and in a ligand-dependent manner with STAT3, while STAT1, STAT4 and STAT5 can also be activated by IL-23. STAT3 activation occurs via tyrosine phosphorylation at Tyr 705 (located in the intracellular domain of the IL-23R) mediated by JAK2. Phosphorylated STAT3 homodimerizes and translocates into the nucleus where it triggers downstream expression of genes like IL-17A, IL-17F, IL-21, IL-22, IL-26 and TNF-α in Th17 cells. (FIG. 7). Ouyang et al., Immunity 28: 454-467 (2008); Altshuler et al., Science 322: 881-888 (2008).

Upon antigenic stimulation, naive CD4+ T-cells differentiate into T-helper (Th) cells Historically, it was understood that this initial differentiation was biphasic, with Th1 cells promoting cellular immunity and protection against instracellular pathogens, and Th2 cells promoting humoral immunity and protecting against extracellular pathogens. Murphy et al., Annu. Rev. Immunol. 18: 451-494 (2000); Glimcher et al., Genes Dev. 14: 1693-1711 (2000). Furthermore, Th1 differentiate from naïve CD4+ T-cells under the influence of IL-12 and IFN-γ, and secrete IFN-γ, while Th2 cells result from IL-4 and IL-13, effecting the cytokines IL-4, IL-5 and IL-13. Th2 cells mediate clearance of extracellular pathogens and IgE-mediated immune responses and allergy. Galli et al., Nature 454: 445-54 (2009).

Recently, it has come to light that naïve CD4+ T cells can also differentiate into a third from of helper cell known as Th17, which results from the influence of cytokines IL-6, TGF-β and IL-23. Th17 cells are crucial in host defense under normal circumstances, but are believed be a key mediator in the establishment and maintenance of autoimmune disease. Th17 cells produce the cytokines IL-17A, IL-17F, IL-21, IL-22, IL-26 and TNF-α Langrish et al., J. Exp. Med. 201(2): 233-240 (2005); Iwakura and Ishigame, J. Clin. Invest. 116(5): 1218-1222 (2006).

IL-17 is a proinflammatory cytokine produced predominantly by activated T-cells. It enhances T cell priming and stimulates fibroblasts, endothelial cells, macrophages, and epithelial cells, resulting in the production of multiple proinflammatory mediators, including IL-1, IL-6, TNF-a, NOS-2, metalloproteases, and chemokines, all of which induce inflammation. Kolls et al., Immunity 21: 467-476 (2004); Nakae et al., Proc. Natl. Acad. Sci. USA 100: 5986-5990 (2003). IL-17 expression is increased in patients with a variety of allergic and autoimmune diseases, such as RA, MS, inflammatory bowel disease (IBD), and asthma, suggestion the contribution of IL-17 to the induction and/or development of such diseases. Supporting this, the involvement of this cytokine in such responses is demonstrated in animal models, autoimmune disorders such as collagen-induced arthritis (CIA), and EAE, animal models for RA and MS, respectively, as well as allergic responses such as contact hypersensitivity, delayed-type hypersensitivity, and airway hypersensitivity were suppressed in IL-17 deficient (IL-17−/−) mice (7,8). Therefore, Th17 cells are likely to play critical roles in the development of autoimmunity and allergic reactions. Oppman et al., Immunity 13: 715-725 (2000), Harrington et al, Nat Immunol 6:1123-1132 (2005), Aggarwal et al., J Biol Chem 278:1910-1914 (2003).

It has further been suggested that IL-23 signaling, but not IL-12/IFN-γ is critical for the development of autoimmune inflammatory diseases. Iwakura et al., J. Clin. Invest. 116 (5): 1218-1222 (2006). Supporting this hypothesis, mice deficient in IL-12p35, IL-12Rβ2, IFN-γ, IFN-γR or STAT1 (all molecules critical in IL-12/IFN-γ signaling) exhibited an increased severity of diseases such as EAE and CIA. Bettelli et al., J. Exp. Med. 200: 79-87 (2004); Hunter, C. A., Nat. Rev. Immunol. 5: 521-531 (2005); McKenzie et al., Trends Immunol. 27: 17-23 (2006). Also, IL-23p19 deficiency, but not IL-12p35 deficiency imposed on IL-10^{-/-} knock outs greatly suppressed the development of colitis. Yen, D. et al., J. Clin. Invest. 116:1310-1316 (2006); Strober et al., Annu. Rev. Immunol. 20: 495-549 (2002). Exogenous IL-23 administration accerlated the onset of colitis in Rag−/− mice engrated with IL-10^{-/-}CD4⁺ T cells. IL-17 production was abolished in IL-23p19^{-/-} mice while IFN-γ and IL-4 production were unaffected. IL-17 and IL-6 expression by anti-CD3 mAb-stimulated memory CD4+ T cells were augmented by IL-23, but not by IL-12. This contrasts with the ability of IL-12 to stimulate native CD4+ T cells. Moreover, treatment with both anti-IL-6 and anti-IL-17 mAbs significantly ameliorated the severity of the intestinal inflammation induced by IL-23-treated Rag^{-/-} mice engrafted with IL-10^{-/-} CD4+ CD45RB^{hi} T cells. This suggests that IL-17 and IL-6 derived from memory T-cells are responsible for the development of intestinal inflammation downstream of IL-23.

While its not entirely clear how IL-23R controls innate immunity through Th17 cells, the fact that others have demonstrated that IL-23R disruptions affects a number of autoimmune diseases such as IBD, psoriasis, alkylosing spondylitis, rheumatoid arthritis and multiple sclerosis indicates the importance of IL-23R in controlling immunity. Abraham et al., Inflamm. Bowel Dis. 15: 1090-1100 (2009). The polymorphisms in IL-23R, such as R381Q could play an important role in the function of the receptor and potentially influence the differentiation and duration of the response of IL-23R expressing cells such as Th17 cells in the host. Accordingly, elevated levels of IL-17F have been reported in the colon of the patients with active CD [Seiderer et al., Inflamm. Bowel Dis. 14: 437-445 (2008)]. In addition, increased IL-22 serum levels were shown in CD and correlate with disease activity and IL-23R wildtype genotype status, while R381Q genotype correlates with decreased levels of serum IL-22 [Schmechel et al., Inflamm. Bowel Dis. 14: 204-212 (2008)].

Under physiological conditions, IL-23 is constitutively expressed in ileal mucosa, and IL-17-producing cells are highly enriched in intestinal tissue. Becker et al., J. Clin. Invest. 112: 693-706 (2003), Ivanov, Cell 126: 1121-33 (2006); Krycek et al., J. Immunol. 178: 6730-33 (2007). The enriched Th-17 cell population in the intestine may be due to a number of factors, including the redident intestinal bacteria Becker, supra. The dynamic balance and coregulation between the IL-23/Th-17 pathway and T_{regs} is continuously in play in the intestinal environment. Intestinal T_{regs} increase in the absence of IL-23, indicating a role for IL-23 in their downregulation. Izcue et al., Immunity 28: 559-70 (2008). Factors enriched in the intestinal environment influence this balance. For example, retinoic acid can inhibit the IL-6-driven induction of Th17 cells and promote anti-inflammatory T_{reg} differentiation. Sun et al., J. Exp. Med. 204: 1775-85 (2007); Mucida et al., Science 317: 256-60 (2007); Elias et al., Blood 111: 1013-20 (2008); Coombes et al., J. Exp. Med. 204: 1757-64 (2007). Consistent with its high level of expression in intestinal tissues, the IL-23/Th-17 pathway is critical in mediating necessary bacterial defenses.

IL-23-driven inflammation is mainly linked to Th17 cells, but recent studies show that IL-23 also has inflammatory effects on immune cells such as innate lymphoid cells and can drive T-cell independent intestinal pathology. Buonocore et al., Nature 464:1371-1375. Lymphoid tissue inducer-like cells (LTi-like cells) constitutively express the IL-23R and were found to be the major source of IL-17 and IL-22 when stimulated with IL-23. In addition to LTi-like cells, δ T cells, which also express IL-23R and able to produce IL-17, IL-21, and IL-22 in response to IL-1β and IL-23 in T cell independent fashion and may mediate autoimmune inflammation. Takatori et al., J Exp Med 206:35-41 (2009). IL-23 signaling has being shown to play a role in regulating allergic asthma through modulation of Th2 cell differentiation by promoting GATA-3 expression. Peng et al., Cell Res 20:62-71. Inflammatory responses associated with IL-23 signaling have being linked to tumor incidence and growth. IL-23 signaling leads to upregulation of the matrix metalloprotease MMP9, and increases angiogenesis but reduces CD8 T-cell infiltration. IL-23 is an important molecular link between tumour-promoting pro-inflammatory processes, thus modulating this pathway may lead to the improved strategies for immune therapy of cancer. Langowski et al., Nature 442:461-465 (2006). In addition, IL-23 plays an important role in suppressing natural or cytokine-induced innate immunity, promoting tumor development and metastases independently of IL-17A. Teng, et al., Proc Natl Acad Sci USA 107:8328-8333. As a result, its clear that while IL-23 signaling may play a dominant role in Th17-inflammation, it is not the only driver of this form of inflammtion, nor is it the only role played by this cytokine pathway in immune function.

2. Detection of IL-23R Loss-of-Function Mutations

In general, methods for detecting IL-23R loss-of-function mutations can be divided into two large groups: (1) methods based on hybridization analysis of polynucleotides, and (2) other methods based on biochemical detection or sequencing of polynucleotides. The most commonly used methods known in the art for the quantification of mRNA expression in a sample include (i) northern blotting, (ii) in situ hybridization (Parker & Barnes, Methods in Molecular Biology 106:247-283 (1999)); (iii) RNAse protection assays (Hod, Biotechniques 13:852-854 (1992)), and (iv) reverse transcription polymerase chain reaction (RT-PCR) (Weis et al., Trends in Genetics 8:263-264 (1992)). Alternatively, (v) anti-nucleic acid antibodies may be employed that can recognize specific duplexes, including (a) DNA duplexes, (b) RNA duplexes, (c) DNA-RNA hybrid duplexes or (d) DNA-protein duplexes. Finally, various methods for determining expression of mRNA or protein include, but are not limited to, (vi) gene expression profiling, (vii) polymerase chain reaction (PCR) including quantitative real time PCR (qRT-PCR), (viii) microarray analysis, such as by using the Affymetrix GenChip technology, (ix) serial analysis of gene expression (SAGE) (Velculescu et al., Science 270:484-487 (1995); and Velculescu et al., Cell 88:243-51 (1997)), (x) MassARRAY, Gene Expression Analysis by Massively Parallel Signature Sequencing (MPSS) (Brenner et al., Nature Biotechnology 18:630-634 (2000)), (xi) proteomics, (xii) immunohistochemistry (IHC), (xiii) gene specific priming, (xiv) promoter methylation analysis, (xv) intron based probes/primers. Preferably, the RNA analyses techniques quantitate the mRNA, such as PCR, specifically qRT-PCR, and microarray analysis.

a. Reverse Transcriptase PCR(RT-PCR)

One of the most sensitive and most flexible quantitative methods is RT-PCR, which can be used to compare mRNA levels in different sample populations, in normal and test sample tissues, to characterize patterns of gene expression, to discriminate between closely related mRNAs, and to analyze RNA structure.

The first step is the isolation of mRNA from a target sample. The starting material is typically total RNA isolated from the tissue affected by or symptomatic of the AID. For example, in the case of the IBD, the tissue sample is a colonic tissue biopsy. Thus, RNA can be isolated from a variety of tissues, including without limitation, the terminal ileum, the ascending colon, the descending colon, and the sigmoid colon. In addition, the colonic tissue from which a biopsy is obtained may be from an inflamed and/or a non-inflamed colonic area.

General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. Total RNA from tissue samples can be isolated using RNA Stat-60 (Tel-Test). RNA prepared from a biopsy can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (Eds) Bioinformatics Methods and Protocols: Methods in Molecular Biology, Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, Tm's between 50° C. and 80° C., e.g. about 50° C. to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

b. Microarrays

Gene expression can also be identified, or confirmed using the microarray technique. Thus, the expression of Il-23R pathway loss-of-function mutant genes can be measured in either fresh or paraffin-embedded tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from biopsy tissue or cell lines derived from cells obtained from a subject having an AID, and corresponding normal tissues or cell lines. For example, RNA can be isolated from a variety of colonic tissues or colonic tissue-based cell lines.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology, or Agilent's Whole Human Genome microarray technology.

c. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

d. MassARRAY Technology

In the MassARRAY®-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

e. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 um diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tissue samples. The mRNA is then extracted, and protein and DNA are removed. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tissues can be isolated, for example, by cesium chloride density gradient centrifugation. After analysis of the RNA concentration, RNA repair and/or amplification steps may be included, if necessary, and RNA is reverse transcribed using gene specific promoters followed by PCR. Peferably, real time PCR is used, which is compatible both with quantitative competitive PCR, where internal competitor for each target sequence is used for normalization, and with quantitative comparative PCR using a normalization gene contained within the sample, or a housekeeping gene for RT-PCR. For further details see, e.g. “PCR: The Polymerase Chain Reaction”, Mullis et al., eds., 1994; and Held et al., Genome Research 6:986-994 (1996). Finally, the data are analyzed to identify the best treatment option(s) available to the patient on the basis of the characteristic gene expression pattern identified in the sample examined.

f. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect IL-23R pathway loss-of-function mutants of the present invention.

g. 5′-multiplexed Gene Specific Priming of Reverse Transcription

RT-PCR requires reverse transcription of the test RNA population as a first step. The most commonly used primer for reverse transcription is oligo-dT, which works well when RNA is intact. However, this technique will not be effective when RNA is highly fragmented. As a result, the invention also employs the use of gene specific primers, which are roughly 20 bases in length with a Tm optimum between about 58° C. and 60° C., and which hybridize to distinct regions of the gene. These primers will also serve as the reverse primers that drive PCR DNA amplification.

An alternative approach is based on the use of random hexamers as primers for cDNA synthesis. However, we have experimentally demonstrated that the method of using a multiplicity of gene-specific primers is superior over the known approach using random hexamers.

h. Promoter Methylation Analysis

A number of methods for quantization of RNA transcripts (gene expression analysis) or their protein translation products are discussed herein. The expression level of genes may also be inferred from information regarding chromatin structure, such as for example the methylation status of gene promoters and other regulatory elements and the acetylation status of histones.

In particular, the methylation status of a promoter influences the level of expression of the gene regulated by that promoter. Aberrant methylation of particular gene promoters has been implicated in expression regulation, such as for example silencing of tumor suppressor genes. Thus, examination of the methylation status of a gene's promoter can be utilized as a surrogate for direct quantization of RNA levels.

Several approaches for measuring the methylation status of particular DNA elements have been devised, including methylation-specific PCR (Herman J. G. et al. (1996) Methylation-specific PCR: a novel PCR assay for methylation status of CpG islands. Proc. Natl. Acad. Sci. USA. 93, 9821-9826) and bisulfite DNA sequencing (Frommer M. et al. (1992). A genomic sequencing protocol that yields a positive display of 5-methylcytosine residues in individual DNA strands. Proc. Natl. Acad. Sci. USA 89, 1827-1831). More recently, microarray-based technologies have been used to characterize promoter methylation status [Chen C. M. (2003) Methylation target array for rapid analysis of CpG island hypermethylation in multiple tissue genomes. Am. J. Pathol. 163, 37-45].

i. Design of Intron-Based PCR Primers and Probes

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

3. Therapeutic Methods of the Invention

The present invention provides therapeutic methods of treating an AID in a subject in need that comprise detecting the presence of an AID in a mammalian subject by the diagnostic methods described herein and then administering to the mammalian subject an appriopriate therapeutic agent. For example, one embodiment of the invention comprising evaluating a sample obtained from an individual for an IL-23R loss-of-function mutant, followed by treating with an IL-23 pathway antagonist if an IL-23R loss-of-function is not present. In some embodiment, the individual is a human. In other embodiments, the individual has AID or is at risk for developing AID. The inflammatory autoimmune disease may be IBD (including ulcerative colitis (UC) or Crohn's disease. In some embodiments, an individual having AID has one that is experiencing or has experienced one or more signs, symptoms, or other indications of AID or has been diagnosed with AID. An individuals having AID may have steroid-refractory and/or steroid dependent IBD (UC or Crohn's disease.) “Steroid-refractory” IBD is IBD which progresses, or worsens, even though steroid is being administered to the subject with IBD. An individual with “steroid-dependent” IBD is dependent on steroid use, and cannot taper or withdraw steroid administration due to persistent symptoms. Severe cases may require surgery, such as bowel resection, strictureplasty or a temporary or permanent colostomy or ileostomy. Alternative medicine treatments for bowel disease exist in various forms, however such methods concentrate on controlling underlying pathology in order to avoid prolonged steroidal exposure or surgical excisement.

In some embodiments, the AID may be T cell dependent to T cell mediated. In some embodiments, the individual with AID has a breakdown in regulatory T-cell mediated tolerance. For example, T cells may be detected at the intestinal lesion site in the individuals with AID. Biopsy samples may be taken from the pathological lesion sites from the individual with AID. The presence of T cells (e.g., CD45Rb may be detected by methods known in the art—e.g., IHB, FACS, etc.). The amount of T cells detected in the biopsy sample may be compared to the amount of T cells in the biopsy sample from a healthy individual or from an intestinal site without inflammation from the same individual.

The administration of an AID therapeutic may result in a clinical response and/or disease remission. As used herein, “clinical response” refers to an improvement in the symptoms of disease. “Disease remission” indicates substantially no evidence of the symptoms of disease. The clinical response or diseaseremission may be achieved within a certain time frame, for example, within or at about 8 weeks from the start of treatment with, or from the initial dose of, the antagonist. Clinical response may also be sustained for a period of time—such as for ≧24 weeks, or ≧48 weeks.

Symptoms associated with IBD, and which may be used to monitor disease progression and or therapeutic dosing of an IL-23 pathway antagonist includes abdominal pain, vomiting, diarrhea, hematochezia (bright red blood in stools), and weight loss. Further tests may be carried out for diagnosing IBD. For example, complete blood cell count, electrolyte panel, liver function tests (LFT), fecal occult blood test, X-rays (including barium enema andupper gastrointestinal series), sigmoidoscopy, colonoscopy, and upper endoscopy may be used. Various scoring system known in the art may be used for quantitatively assessing severity of the disease.

For the prevention of treatment of disease, the appropriate dosage of an active agent (i.e., an IL-27 antagonist), will depend on the type of disease to be treated, the severity and coruse of the disease, whether the agent is administered for preventive or therapeutic purpose, previous therapy, the patient's clinical history and response to the agent, and the discretion of the attending physician. The particular dosage regimen, i.e., dose, timing and repetition, will depend on the particular individual and that individual's medical history as assessed by a physician.

Methods of the present invention are useful for treating, ameliorating or palliating the symptoms of IBD (such as ulcerative colitis, or Crohn's disease) in an individual, or for improving the prognosis of an individual suffering from IBD. The quality of life in individuals suffering from IBD may be improved, and the symptoms of BID may be reduced or eliminated following treatment. For example, weight loss associated with IBD may be reduced and/or eliminated. Methods of the present invention are also useful for delaying development of or preventing IBD in and individual at risk for developing IBD.

a. Combination Therapies

The methods described herein further contemplates combining the various AID therapeutic agents that may be suitable for use in the method described herein (see St Clair Jones, Hospital Pharmacist, May 2006, Vol. 13; pages 161-166, hereby incorporated by reference in its entirety).

Treatment of the autoimmune inflammatory diseases, including IBD, of the present invention comprises evaluating a patient for the presence of an IL-23R LOF mutant followed by the administration of the appropriate therapeutic optionally in combination with a second medicament or treatment regimen. The type of such second medicament depends on various factors, including the type of AID, the severity of AID, the condition and age of the subject, the type and dose of the first medicament employed. In one embodiment, the AID therapeutic agent is synonymous with “other than IL-23 pathway antagonist,” as defined herein. Specific examples include: (1) Aminosalicylates/anti-inflammatory drugs—sulfasalazine (Azulfidine®), mesalamine (Asacol®, Rowasa®, Pentasa®), olsalazine (Depentum®) and balsalazide (Colazal®); (2) Corticosteroids—methylprednisolone, hydrocortisone, prednisone, prednisolone, budesonide, dexamethasone; (3) Immune system suppressors—azathioprine) (Imuran® and mercaptopurine)(Purinethol®. (4) Antibodies, and antibody derivatives (biologics)—(i) anti-TNF-α antibody: infliximab/Remicade®, adalimumab/Humira®, certolizumab pegol/Cimzia®, and (ii) serum immunoglobulins—Sandimmune®, natalizumab/Tysabri®; (5) Antibiotics—metronidazole/Flagyl®, ciprofloxacin/Cipro®, Neoral®; (6) Anti-metabolic agents—methotrexate/Rheumatrex®; (7) Palliative therapies—anti-diarrheals, laxatives, pain relievers, iron supplements, nutrition, vitamin B-12, calcium and vitamin D, TNF antagonist (non-biologic, thalidomide), cyclosporine A, nicotine patch, butyrate enema, heparin.

All of these second medicaments may be used in combination with each otheror by themselveswith the first medicament, so that the expression “second medicament” as used herein does not mean it is the only medicament besides the first one, respectively. Thus, the second medicament need not be one drug, but may constitute or comprise more than one drug.

The least toxic AID therapeutic agents which patients with IBD are typically treated with for are the aminosalicylates. Sulfasalazine (Azulfidine), typically administered four times a day, consists of an active molecule of aminosalicylate (5-ASA) which is linked by an azo bond to a sulfapyridine. Anaerobic bacteria in the colon split the azo bond to release active 5-ASA. However, at least 20% of patients cannot tolerate sulfapyridine because it is associated with significant side-effects such as reversible sperm abnormalities, dyspepsia or allergic reactions to the sulpha component. These side effects are reduced in patients taking olsalazine. However, neither sulfasalazine nor olsalazine are effective for the treatment of small bowel inflammation. Other formulations of 5-ASA have been developed which are released in the small intestine (e.g. mesalamine and asacol). Normally it takes 6-8 weeks for 5-ASA therapy to show full efficacy. Patients who do not respond to 5-ASA therapy, or who have a more severe disease, are prescribed corticosteroids. However, this is a short term therapy and cannot be used as a maintenance therapy. Clinical remission is achieved with corticosteroids within 2-4 weeks, however the side effects are significant and include Cushing goldface, facial hair, severe mood swings and sleeplessness. The response to sulfasalazine and 5-aminosalicylate preparations is poor in CD, fair to mild in early ulcerative colitis and poor in severe UC. If these agents fail, powerful immunosuppressive agents such as cyclosporine, prednisone, 6-mercaptopurine or azathioprine (converted in the liver to 6-mercaptopurine) are typically tried. For CD patients, the use of corticosteroids and other immunosuppressives must be carefully monitored because of the high risk of intra-abdominal sepsis originating in the fistulas and abscesses common in this disease. Approximately 25% of IBD patients will require surgery (colectomy) during the course of the disease.

The second treatment regimen may also a surgical procedure. For example, in IBD surgical procedures include, without limitation, a bowel resection, anastomosis, a colectomy, a proctocolectomy, and an ostomy, or any combination thereof.

In addition to pharmaceutical medicine and surgery, nonconventional treatments for AID such as nutritional therapy may be used. For example, Flexical®, a semi-elemental formula, has been shown to be as effective as the steroid prednisolone. Sanderson et al., Arch. Dis. Child. 51:123-7 (1987). However, semi-elemental formulas are relatively expensive and are typically unpalatable—thus their use has been restricted. Nutritional therapy incorporating whole proteins has also been attempted to alleviate the symptoms of IBD. Giafer et al., Lancet 335: 816-9 (1990). U.S. Pat. No. 5,461,033 describes the use of acidic casein isolated from bovine milk and TGF-2. Beattie et al., Aliment. Pharmacol. Ther. 8: 1-6 (1994) describes the use of casein in infant formula in children with IBD. U.S. Pat. No. 5,952,295 describes the use of casein in an enteric formulation for the treatment of IBD. However, while nutrional therapy is non-toxic, it is a palliative treatment and does not treat the underlying cause of the disease.

In one aspect, the invention provides methods for treating or preventing an AID, the methods comprising detecting the presence of an AID in a subject and administering an effective amount of an AID therapeutic agent to the subject. It is understood that any suitable AID therapeutic agent may be used in the methods of treatment, including aminosalicylates, corticosteroids, and immunosuppressive agents as discussed herein.

In any of the methods herein, one may administer to the subject or patient along with a single AID therapeutic agent discussed herein an effective amount of a second medicament (where the single AID therapeutic agent herein is a first medicament), which is another active agent that can treat the condition in the subject that requires treatment. For instance, an aminosalicylate may be co-administered with a corticosteroid, an immunsuppressive agent, or another aminosalicylate. The type of such second medicament depends on various factors, including the type of AID, its severity, the condition and age of the patient, the type and dose of first medicament employed, etc.

Such treatments using first and second medicaments include combined administration (where the two or more agents are included in the same or separate formulations), and separate administration, in which case, administration of the first medicament can occur prior to, and/or following, administration of the second medicament. In general, such second medicaments may be administered within 48 hours after the first medicaments are administered, or within 24 hours, or within 12 hours, or within 3-12 hours after the first medicament, or may be administered over a pre-selected period of time, which is preferably about 1 to 2 days, about 2 to 3 days, about 3 to 4 days, about 4 to 5 days, about 5 to 6 days, or about 6 to 7 days.

The first and second medicaments can be administered concurrently, sequentially, or alternating with the first and second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the express “second medicament” as used herein does not mean it is the only medicament besides the first medicament, respectively. Thus, the second medicament need not be one medicament, but may constitute or comprise more than one such drug. These second medicaments as set forth herein are generally used in the same dosages and with administration routes as the first medicaments, or about from 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

Where the methods of the present invention comprise administering one or more IBD therapeutic agent to treat or prevent an IBD, it may be particularly desirable to combine the administering step with a surgical procedure that is also performed to treat or prevent the IBD. The IBD surgical procedures contemplated by the present invention include, without limitation, a bowel resection, anastomosis, a colectomy, a proctocolectomy, and an ostomy, or any combination thereof. For instance, an IBD therapeutic agent described herein may be combined with a colectomy in a treatment scheme, e.g. in treating an IBD. Such combined therapies include and separate administration, in which case, administration of the IBD therapeutic agent can occur prior to, and/or following, the surgical procedure.

Treatment with a combination of one or more IBD therapeutic agents; or a combination of one or more IBD therapeutic agents and a surgical procedure described herein preferably results in an improvement in the signs or symptoms of an IBD. For instance, such therapy may result in an improvement in the subject receiving the IBD therapeutic agent treatment regimen and a surgical procedure, as evidenced by a reduction in the severity of the pathology of the IBD.

The AID therapeutic agent(s) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

The AID therapeutic agent(s) compositions administered according to the methods of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The first medicament(s) need not be, but is optionally formulated with one or more additional medicament(s) (e.g. second, third, fourth, etc. medicaments) described herein. The effective amount of such additional medicaments depends on the amount of the first medicament present in the formulation, the type of disorder or treatment, and other factors discussed above. These are generally used in the same dosages and with administration routes as used hereinbefore or about from 1 to 99% of the heretofore employed dosages.

For the prevention or treatment of an AID, the appropriate dosage of an AID therapeutic agent (when used alone or in combination with other agents) will depend on the type of disease to be treated, the type of AID therapeutic agent(s), the severity and course of the disease, whether the AID therapeutic agent is administered for preventive or therapeutic purposes, previous therapy, the patient's clinical history and response to the AID therapeutic agent, and the discretion of the attending physician. The AID therapeutic agent is suitably administered to the patient at one time or over a series of treatments. Depending on the type and severity of the disease, about 1 ug/kg to 15 mg/kg (e.g. 0.1 mg/kg-10 mg/kg) of AID therapeutic agent is an initial candidate dosage for administration to the patient, whether, for example, by one or more separate administrations, or by continuous infusion. One typical daily dosage might range from about 1 ug/kg to 100 mg/kg or more, depending on the factors mentioned above. For repeated administrations over several days or longer, depending on the condition, the treatment is sustained until a desired suppression of disease symptoms occurs. One exemplary dosage of the AID therapeutic agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the AID therapeutic agent). An initial higher loading dose, followed by one or more lower doses may be administered. An exemplary dosing regimen comprises administering an initial loading dose of about 4 mg/kg, followed by a weekly maintenance dose of about 2 mg/kg of the AID therapeutic agent. However, other dosage regimens may be useful. The progress of this therapy is easily monitored by conventional techniques and assays.

4. Production of Antibodies

The present invention further provides antibodies. Exemplary antibodies include polyclonal, monoclonal, humanized, bispecific, and heteroconjugate antibodies. As discussed herein, the antibodies may be used in the diagnostic methods for AID, and in some cases in methods of treatment of AID, including antibodies that target other than an IL-23R pathway antagonist.

(a) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(b) Monoclonal Antibodies

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pliickthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(c) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. An example of a humanized antibody used to treat IBD is infliximab (Remicade®), an engineered murine-human chimeric monoclonal antibody. The antibody binds the cytokine TNF-alpha and prevents it from binding its receptors to trigger and sustain an inflammatory response. Infliximab is used to treat both CD and UC.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of the humanized antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

(d) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

(e) Antibody Fragments

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(f) Bispecific Antibodies

Bispecific antibodies are antibodies that have binding specificities for at least two different epitopes. Exemplary bispecific antibodies may bind to two different epitopes of an AID marker protein. Bispecific antibodies may also be used to localize agents to cells which express an AID marker protein.

These antibodies possess an AID marker-binding arm and an arm which binds an agent (e.g. an aminosalicylate). Bispecific antibodies can be prepared as full length antibodies or antibody fragments (e.g. F(ab′)2 bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

(g) Other Amino Acid Sequence Modifications

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in the following table, or as further described below in reference to amino acid classes, may be introduced and the products screened.

TABLE 2

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | tip; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)): non-polar: Ala (A), Val (V), Leu (L), Ile (I), Pro (P), Phe (F), Tip (W), Met (M); uncharged polar: Gly (G), Ser (S), Thr (T), Cys (C), Tyr (Y), Asn (N), Gln (O); acidic: Asp (D), Glu (E); and basic: Lys (K), Arg (R), His(H).

Alternatively, naturally occurring residues may be divided into groups based on common side-chain properties: hydrophobic: Norleucine, Met, Ala, Val, Leu, Ile; neutral hydrophilic: Cys, Ser, Thr, Asn, Gln; acidic: Asp, Glu; basic: His, Lys, Arg; residues that influence chain orientation: Gly, Pro; and aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and an AID marker protein. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Engineered antibodies with three or more (preferably four) functional antigen binding sites are also contemplated (U.S. Published Patent Application No. US2002/0004587 A1, Miller et al.).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

5. Kits of the Invention

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for AID. Such kits may optionally contain reagents for the extraction of RNA from samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

Having described the invention, the same will be more readily understood through reference to the following Examples, which is provided by way of illustration, and is not intended to limit the invention in any way.

### Example

### Introduction—

Crohn's disease (CD) is an inflammatory bowel disease (IBD), which occurs due to aberrant response of the gut-associated lymphoid tissue to bacterial and dietary antigen. Genome-wide association studies (GWAS) in several populations have demonstrated strong associations of the IL-23R gene with Crohn's disease (CD) and psoriasis, suggesting that perturbation of the IL-23 signaling pathway is relevant to the pathophysiology of these diseases. One particular variant, R381Q (rs11209026), confers strong protection from CD (odds ratio=0.26, and case-control cohorts allele frequency is 0.15 to 0.43), but its mechanism of action remains unknown. We investigated the effects of the R381Q IL-23R variant in transfected cell lines and in primary cells from donors carrying IL-23R^{R381} and IL-23R^{Q381} haplotypes. IL-23R^{Q381} was associated with strikingly diminished IL-23R surface expression. T-cells from donors carrying at least one IL-23R^{Q381} allele showed decreased STAT3 phosphorylation upon stimulation with IL-23. This was not due to a direct effect of the R381Q variant on IL-23R signaling capacity, because cell lines artificially selected to express similar levels of either IL-23R^{R381} or IL-23R^{Q381} at the surface did not differ in IL-23R-mediated signaling. Our data indicate that IL-23R^{Q381} is a hypomorphic IL-23R allele that results in reduced cell surface expression and protection from CD and other immune diseases. Our study is the first to show conclusively that IL-23R^{Q381} is a loss-of-function allele, further strengthening the implication from GWAS results that the IL-23 pathway is pathogenic in human disease. This data provides an explanation for the protective role of R381Q in CD and suggestive of a new treatment intervention for autoimmune inflammatory disorders.

The interleukin 23 receptor (IL-23R) gene shows association with Crohn's disease (CD)⁶⁻¹⁴⁾, psoriasis⁽¹⁵⁾ and ankylosing spondylitis⁽¹⁶⁾ in multiple independent populations. The R381Q coding variant (rs11209026, c.1142G>A) of IL-23R was found at lower frequencies in disease-affected individuals and is therefore protective. However, the mechanism by which IL-23R^{Q381} confers protection is unresolved and remains a question of key importance, because as of yet there is no conclusive evidence that IL-23 signaling is pathogenic in human disease; the possibility that it is part of an ineffective protective response in heavily inflamed tissue can not be formally ruled out.

IL-23R is most highly expressed on activated T cells, particularly of the Th17 subtype, and at lower levels on monocytes, macrophages and dendritic cells. IL-23R pairs with IL12Rβ1 to confer IL-23 responsiveness on cells expressing both receptor subunits^{(17),(18)}. IL-23R associates constitutively with JAK2 and, in a ligand-dependent manner, with STAT3. STAT1, STAT4 and STAT5 can also be activated by IL-23⁽³⁰⁾. Several studies suggest that IL-23R is also a key player in proliferation and survival of Th17 cells, which have been implicated in inflammatory and autoimmune disorders⁽¹⁸⁻²⁰⁾. Studies in intestinal tissues have shown that IL-17F and IL-22 mRNA expression (induced via IL-23 signaling) are significantly increased in inflamed colonic lesions in CD compared to uninflamed biopsies and IL-22 is associated with a higher expression of inflammatory mediators. Seiderer et al., Inflamm. Bowel. Dis. 2008; 14:437-45; Brand et al., Am. J. Gasterointest. Liver Physiol. 2006; 290:G827-38. In addition, increased IL-22 serum levels were reported in CD patients and correlated with disease activity and IL-23R genotype status. Interestingly, the R381Q allege correlates with slightly decreased serum levels of IL-22 24, implying that this variant leads to reduced cytokine production.

The potential involvement of IL-23R^{Q381} in the pathogenesis of CD and other autoimmune disorders prompted us to conduct an in-depth functional analysis of this protective variant.

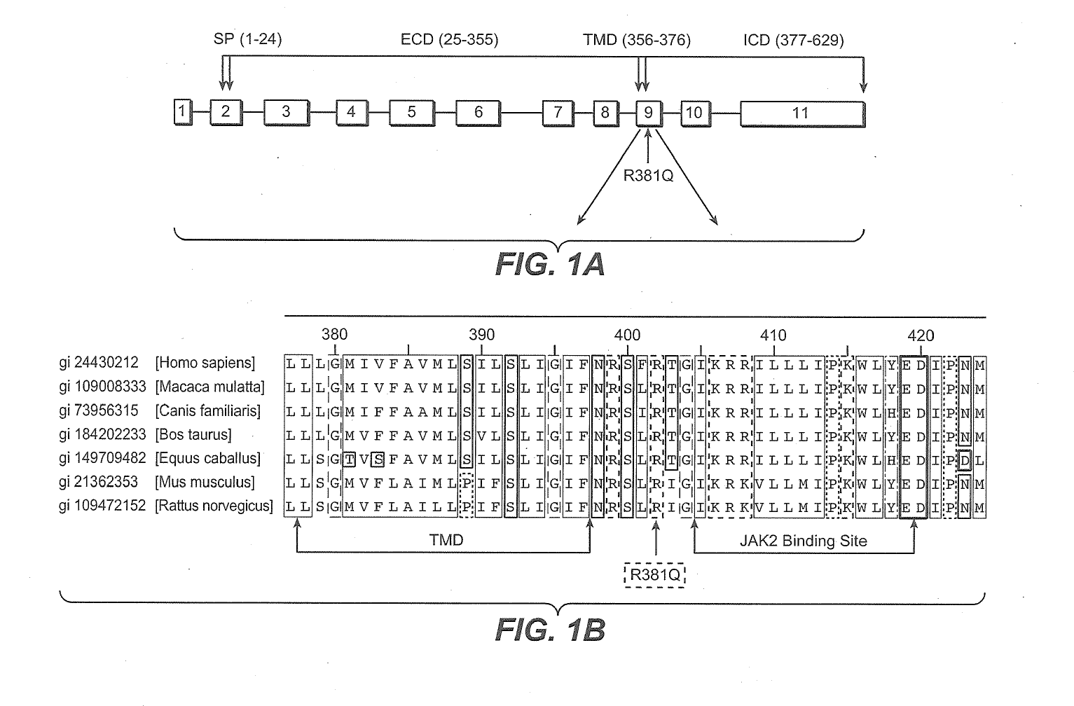

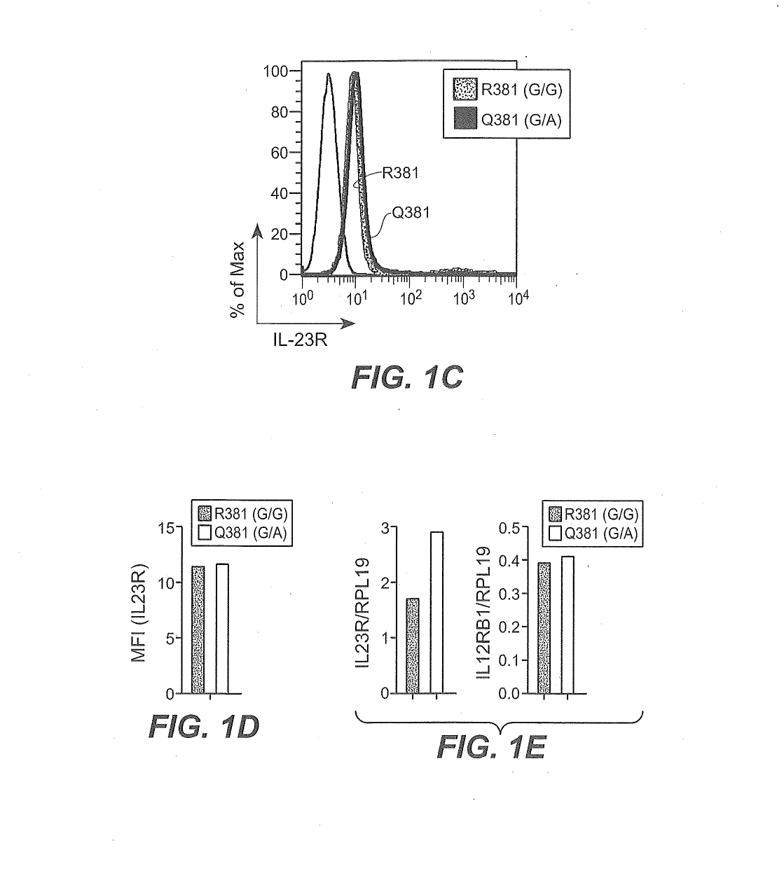

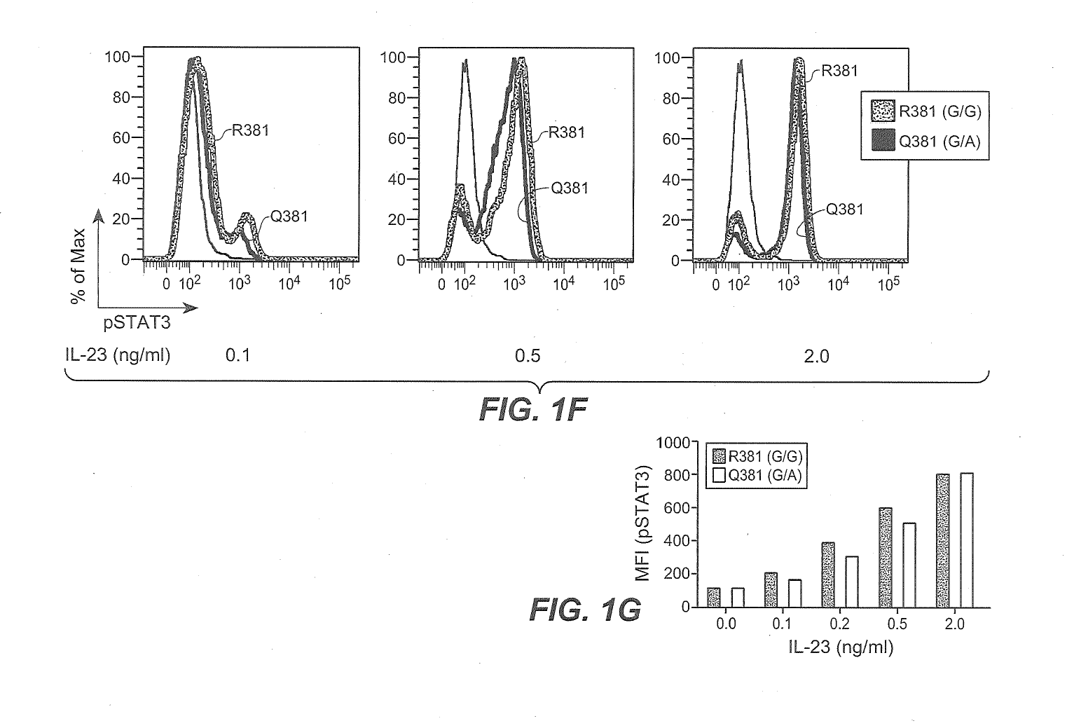

The R381Q polymorphism is located between the transmembrane domain and the putative Jak2 binding site in the cytoplasmic portion of IL-23R, and is absolutely conserved across different species (FIGS. 1A and 1B). By virtue of this location, it could interfere with surface localization of the IL-23R⁽²¹⁾ and/or signal transduction⁽²²⁾. Conversely, it is unlikely to interfere with ligand binding, nor will it compromise our interpretation of flow cytometry experiments with IL-23R specific antibodies.

We first introduced cDNAs encoding IL-23R^{R381} or IL-23R^{Q381} into factor dependent BaF3(23) cells that had previously been stably transfected with the IL12R¹³¹ chain. To analyze IL-23R surface expression, we used a proprietary biotinylated mouse anti human IL-23R antibody clone 20G3.4 in combination with streptavidin-PE and/or the same antibody directly conjugated to FITC. To verify whether IL-23R^{Q381} per se affects signaling, we identified single BaF3 clones with equivalent cell surface expression of IL-23R^{R381} and IL-23^{Q381} (FIGS. 1C and 1D). Interestingly, we consistently observed that slightly higher expression of IL-23R^{Q381} mRNA was required to effect equivalent IL-23R cell surface expression, indicating that the R381Q polymorphism might interfere with translation and/or cell surface locatlization of the protein (FIG. 1E). Furthermore, upon stimulation, IL-23R^{Q381} expressing clones shows slightly decreased pSTAT3 responses when stimulated with limited concentrations of IL-23 (FIGS. 1F and 1G), suggesting that the R381Q conversion impairs the ability of IL-23R to signal efficiently.

We next used a more physiologically relevant in vitro system to study the effects of the R381Q polymorphism by generating untransformed polyclonal T cell lines for donors carrying either IL-23R^{R381} or IL-23R^{Q381}. We genotyped 138 healthy donors and identified eighteen IL-23R^{Q381} heterozygous individuals and one homozygous individual. The calculated allege frequency of 7.1% is R381Q is consistent with the published estimates of 7.0% [Duerr et al., Science 314: 1461-1463 (2006)], 7.3% [Roberts et al., Am. J. Gastroenterol. 2007; 102:2754-61] and 6.0% [Newman et al., Clin. Gasterolenterol. 2009; 43:444-7]. We generated T cell lines from five IL-23R^{R381}, four IL-23R^{Q381} heterozygous, and one IL-23R^{Q381} homozygous donors. All donors were Caucasian ages 25 to 65.

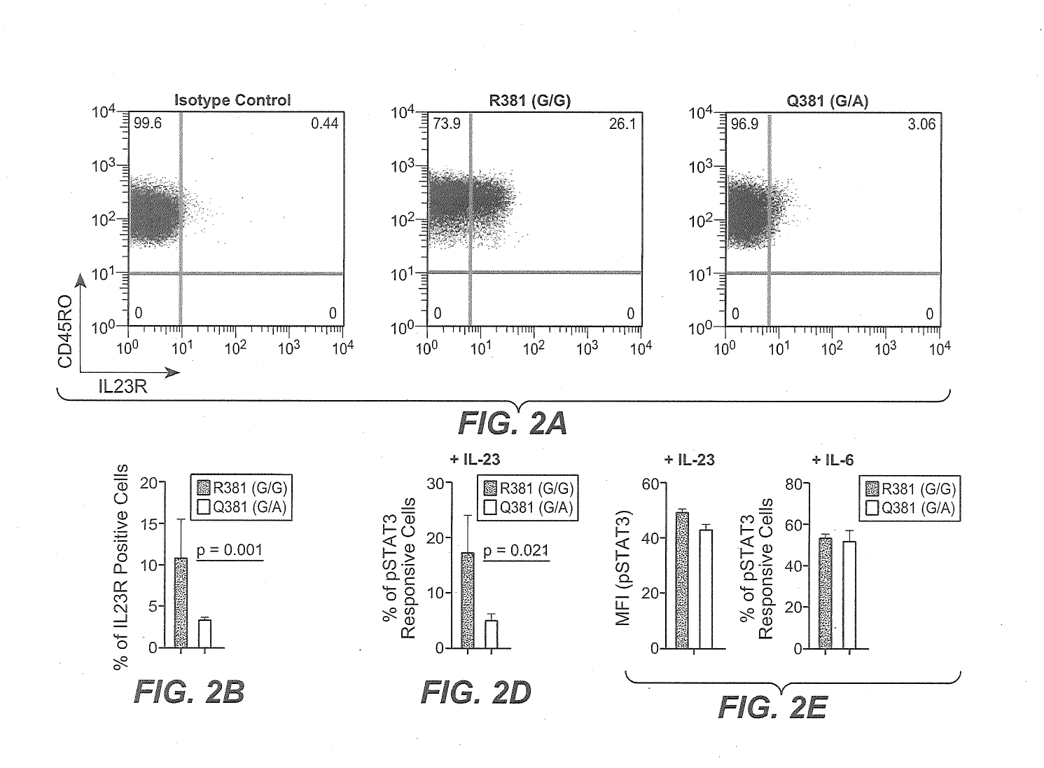

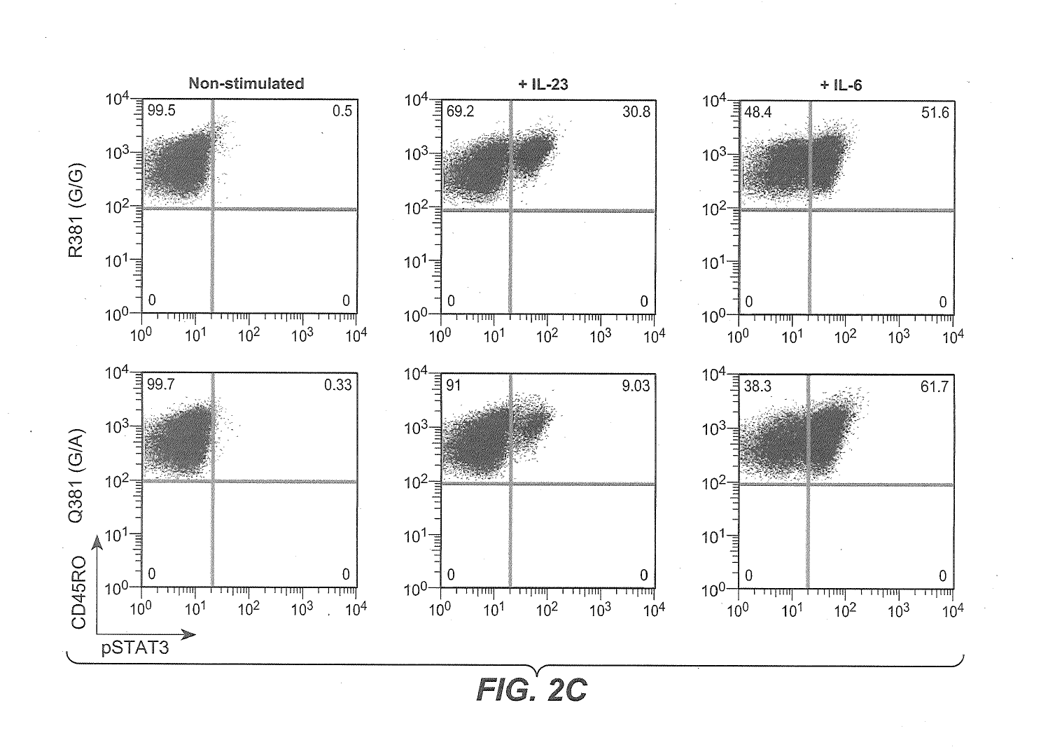

We observed no obvious differences in proliferation rates between these T cell lines during in vitro expansion (data not shown). However, flow cytometry analysis performed after six days of in vitro stimulation with feeder mixture (see Methods), revealed significantly diminished population of IL-23R positive T cells from IL-23R^{Q381} positive donors compared to the IL-23R^{Q381} counterparts (FIGS. 2A and 2B). When stimulated with IL-23 we observed fewer STAT3 positive cells in the IL-23R^{Q381} samples (FIG. 2C-D). In addition, the MFI of IL-23 stimulated pSTAT3 positive cells was decreased (FIG. 2E), suggesting that not only was the strength of the IL-23 response for any given cell decreased by the R381Q polymorphism, there was also a specific paucity of IL-23 respsonsive T-cells in the samples of IL-23R^{Q381} positive individuals. By comparison, IL-6 elicited pSTAT3 responses were independent of the IL-23R genotype, demonstrating that IL-23R^{Q381} positive cells are intrinsically capable of full STAT3 activation (FIGS. 2C and 2D).

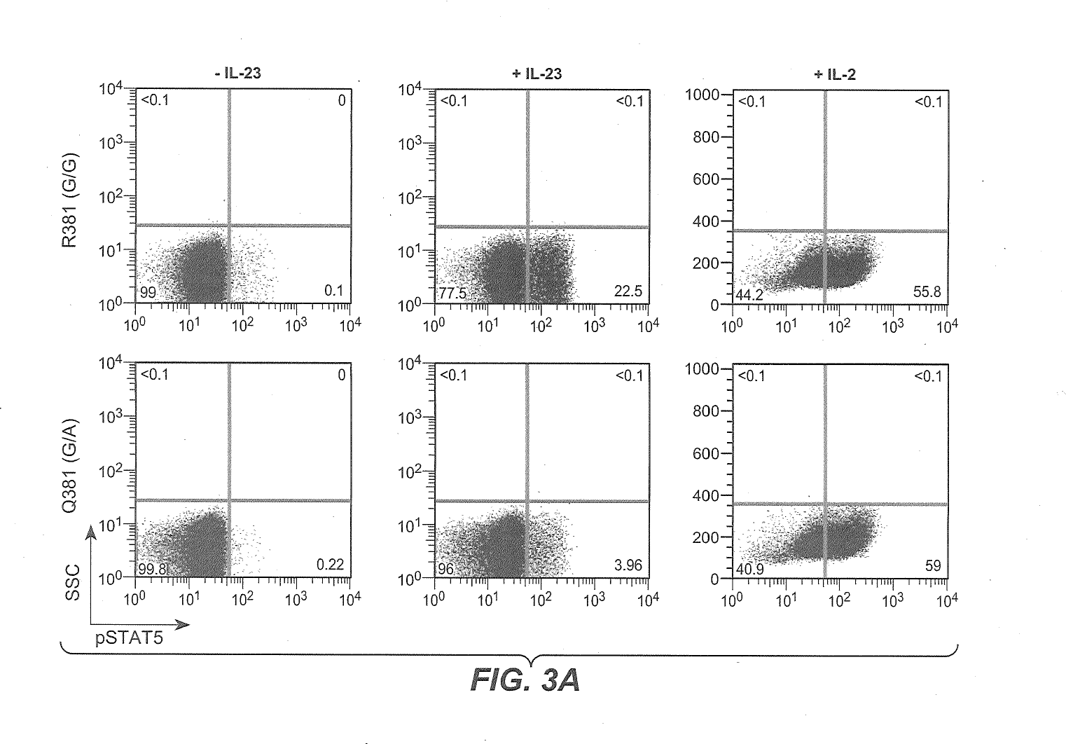

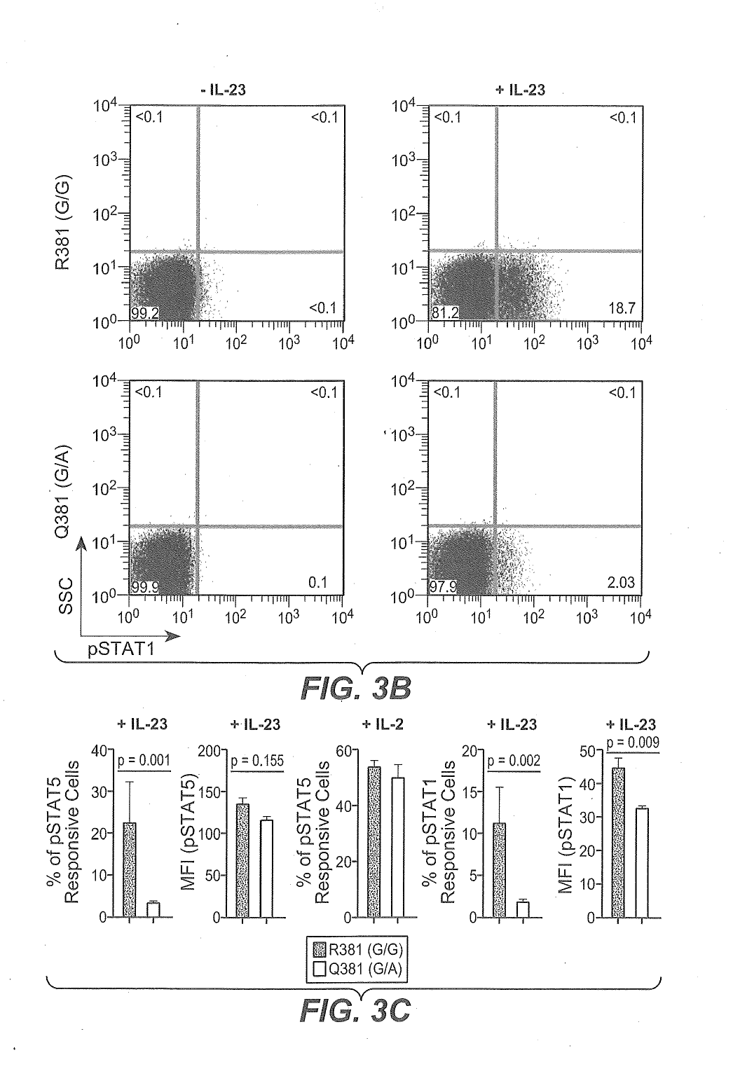

To further support this interpretation, we examined IL-23 induced STAT1 and STAT5 phosphorylation. To reduce background levels of pSTAT5 elicited by endogenously produced IL-2, we added a neutralizing monoclonal antibody anti-human IL-2 antibody to the cultures while stimulating T cells with IL-23. We observed significantly decreased levels of both STAT5 (FIGS. 3A and 3C) and pSTAT1 (FIGS. 3B and 3C) in IL-23R^{Q381} positive T cells compared to the IL-23R^{Q381} positive lines. The MFI of IL-23 stimulated pSTAT5 positive cells was slightly decreased and pSTAT1 positive cells significantly decreased in agreement with the defect observed in STAT 3 phosphorylation. On the other hand, when stimulated with IL-2, pSTAT5 levels were equivalent between IL-23R^{R381} and IL-23R^{Q381} cell lines (FIGS. 3A and 3C).

In addition to the T cell lines generated from IL-23R^{R381} and IL-23R^{Q381} donors, we analyzed freshly isolated PBMCs from the same donors. IL-23R expression is known to be activation dependent [Wilson et al., Nat. Immunol. 8: 950-957 (2007)], and indeed we could not detect any IL-23R expression on freshly isolated PBMC (not shown). Therefore, we next stimulated total PBMCs with agonist antibodies directed against CD3 and CD28 for 72 hours and then analyzed IL-23R surface expression. After stimulation, we clearly observed IL-23R expression on the IL-23R^{R381}T cells, while the IL-23R^{Q381} T cells has slightly, but not significantly decreased number of IL-23R positive cells (FIGS. 7A and 7B). We also observed diminished levels of pSTAT3 positive IL-23R^{R381} CD4+ cells, compared to IL-23R^{Q381} CD4+ cells, when we stimulated whole blood with IL-23. IL-6 stimulation resulted in similar responses (FIGS. 7C and 7D), confirming our data from T cell lines and IL-23R tranduced BaF3 clones. Interestingly, the CD4− population did not show any defect in STAT3 phosphorylation (FIG. 7C). This observation is consistent with the specific decrease of IL-23 responsive T-cells in IL-23R^{Q381} donors.

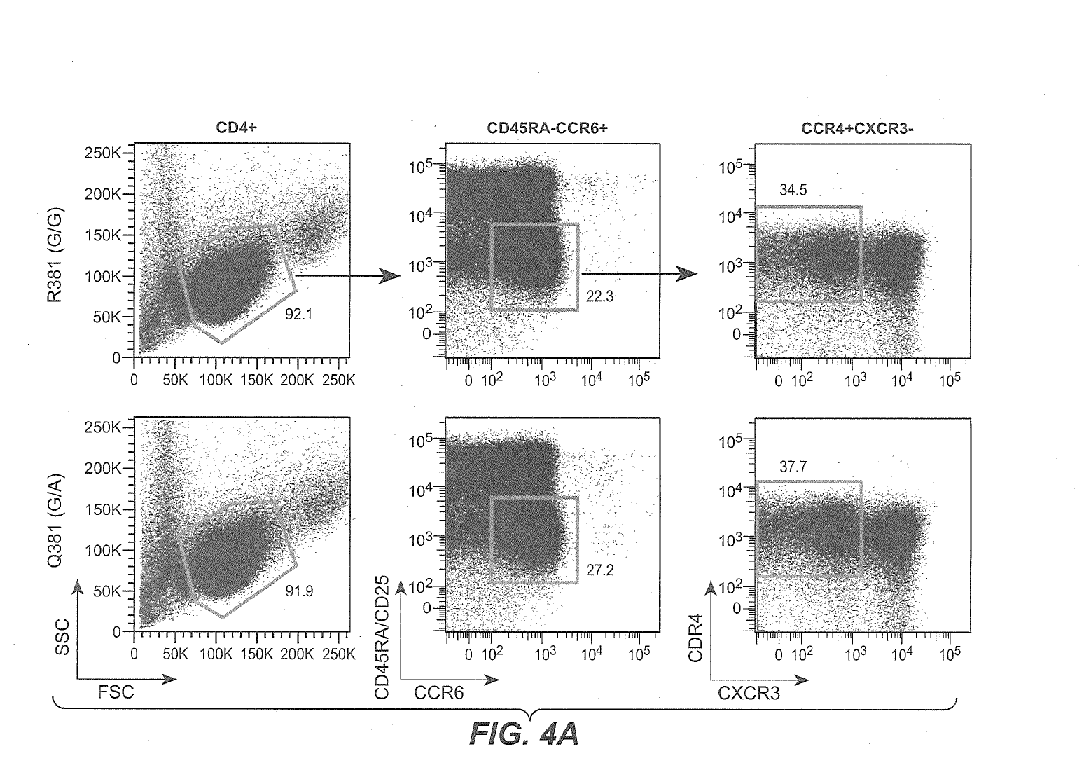

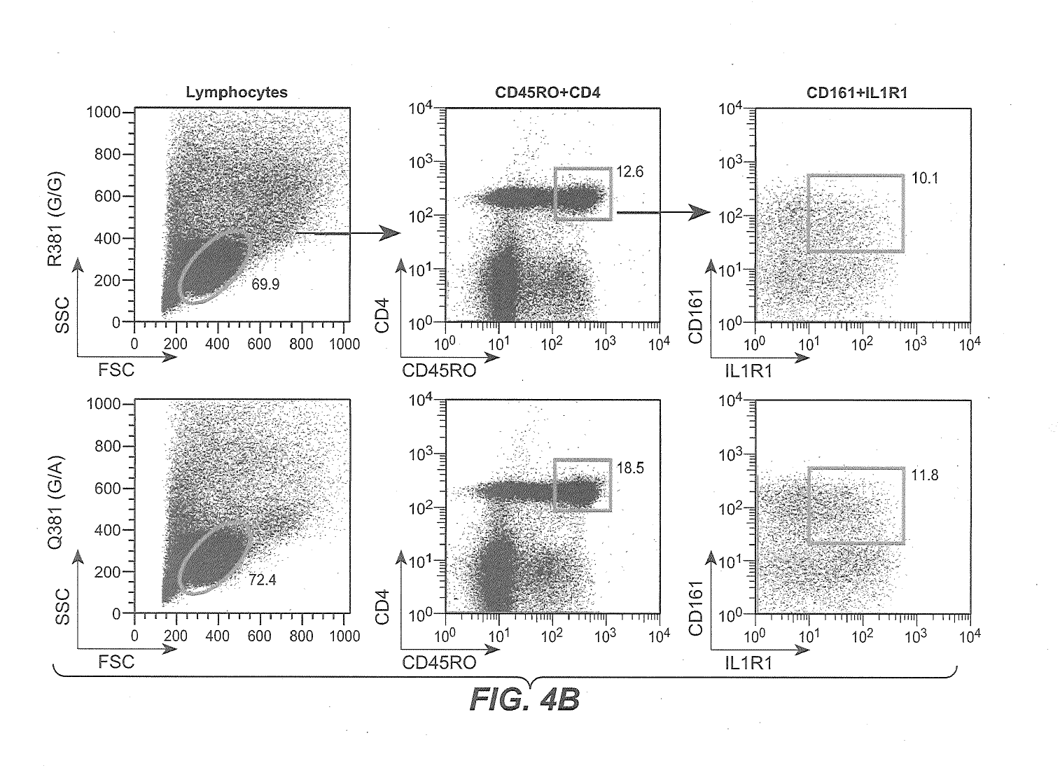

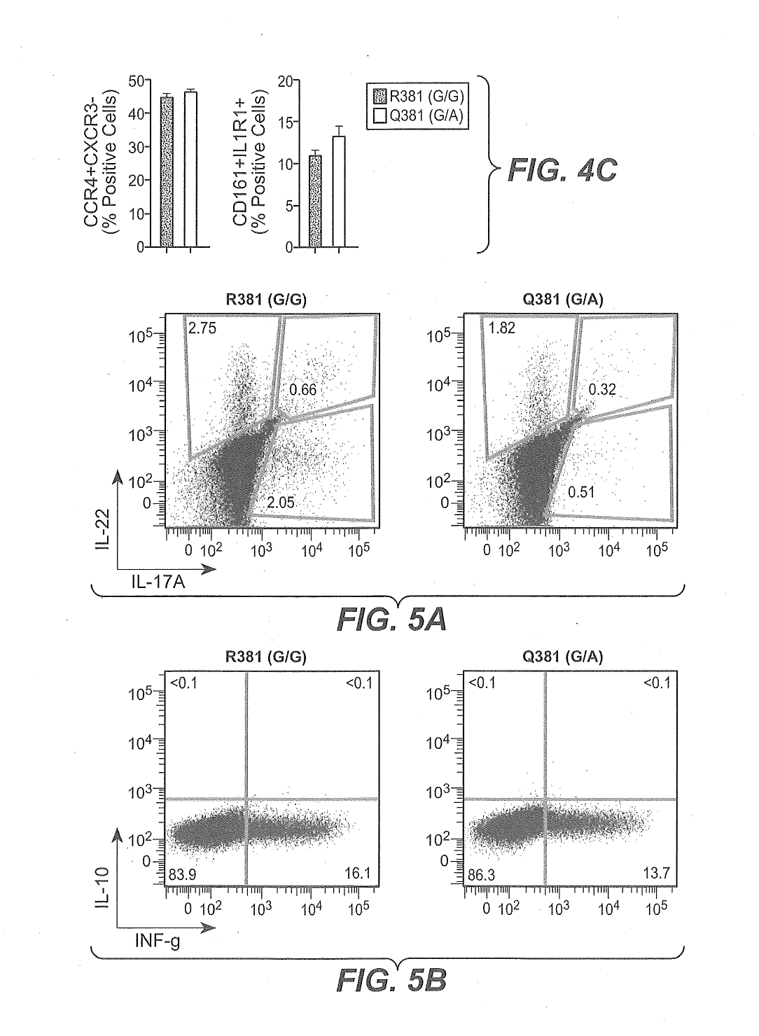

IL-23 is required for the production of IL-17 by human Th17 cells. Thus, IL-23R^{Q381} could potentially influence the differentiation and duration of the Th17 response. To explore this possibility, we first performed multicolor flow cytometry analysis on freshly isolated PBMCs from IL-23R^{R381} and IL-23R^{Q381} positive donors to analyze Th17 cells. Acosta-Rodriguez et al., Nat. Immunol. 8: 639-646 (2007). However, when we gated on this subset and additional markers such as IL1R1 and CD161, which has been shown to be a gut homing Th17 cell marker [Kleinschek et al., J. Exp. Med. 206: 525-534 (2009)], we did not observe significant differences between IL-23R^{R381} and IL-23R^{Q381} positive donors (FIG. 4).

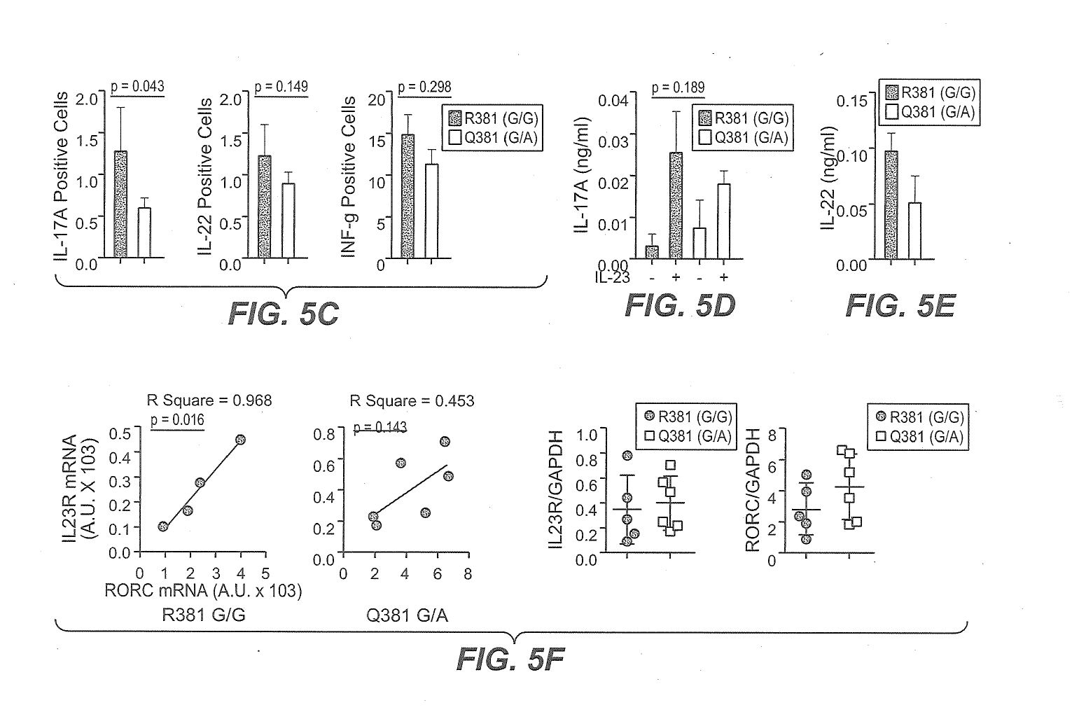

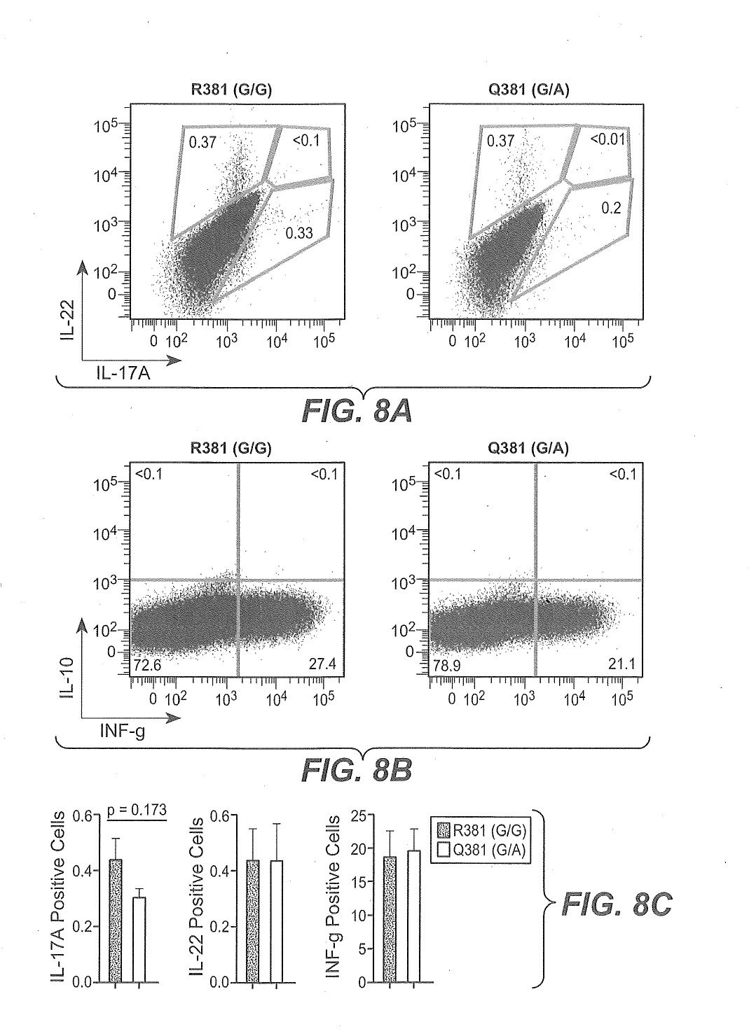

To investigate the functional impact of the IL-23R^{Q381} in terms of cytokine secretion, we used stimulated T cell lines from IL-23R^{R381} and IL-23R^{Q381} donors to measure the production of cytokines by intracellular staining (FIG. 5A-C). We observed slightly, but not significantly decreased levels of cytokines IL-23R^{Q381} positive donors. Similar results were observed when fresh PBMCs from IL-23R^{R381} and IL-23R^{Q381} positive donors were stimulated (FIG. 8). Serum IL-22 levels were determined using ELISA, and we again observed a slight, but not significant, trend in decreased IL-22 production in IL-23R^{Q381} donors compared to IL23R^{R381} donors, in agreement with previously published data. Schmechel et al., Inflamm. Bowel Dis. 2008; 14:204-12. However, we did observe significantly decreased numbers of IL-17A+ cells by intracellular staining (FIGS. 5A and 5C) and slightly diminished IL-23 dependent IL-17 induction in supernatents from stimulated PBMCs (FIG. 5D). Finally, we extracted RNA from fresh PBMCs and analyzed IL-23R and RORC (a Th17 marker) mRNA expression by real-time PCR. We did not observe a significant correlation between IL-23R and RORC mRNA levels, though the correlation between these two transcripts was more apparent in the IL-23^{R381} cells than in the IL-23R^{Q381} cells (FIG. 5F), suggesting that the variant might have some role in production or processing of the mRNA of IL23R itself.

In summary, we used several in vitro systems to demonstrate conclusively that R381Q polymorphism results in a decreased population of IL-23 responsive cells, manifesting in diminished IL-23 dependent activation of all STATs known to be associated with IL-23 signaling. Several mechanisms might contribute to this observation, including diminished cell surface expression relative to a given amount of IL-23R mRNA, and a reduced capacity of IL-23R^{Q381} to activate STAT proteins. Furthermore, our results are consistent with a recent report by Gallagher et al., J. Immunol. 184: 151-154 (2010), suggesting that the rs11209026 SNP affects splicing in such a way that soluble IL-23R would be generated, which subsequently blocks the differentiation of IL-23 responsive T-cells.

One of the major advantages of our study was the availability of healthy donors willing to donate blood repeatedly for our primary T-cell studies. It should be noted that while the analyis described herein was limited to T cells, the protective effect of IL-23R^{Q381} is likely to be comediated by other IL-23 responsive cell types (e.g., dendritic cells) in vivo. Parham et al., J. Immunol. 168: 5699-5708 (2002). We did not detect significant differences in cytokine levels by ICS and ELISA between IL-23R^{R381} and IL-23R^{Q381} positive donors in peripheral blood; however, this is unlikely to be a physiologically relevant tissue in Crohn's disease pathogenesis. Indeed, a recent study (Veny et al., Aliment Pharmacol. Ther. 2009) demonstrated that only patients with late active CD showed increased IL-17 production, as well as a significantly higher percentage of IL-17+CD4+ cells in blood, but not patients with early signs of the disease or patients in remission, even though an increased IL-17 gene transcription is common to early and late CD mucosa, indicating that exacerbated Th17 responses in the peripheral blood appear only in late disease.

It has been demonstrated in humans, as well as in animal models, that gut flora plays a critical role in the development of intestinal Th17. Ivanov, I I et al., Cell Host Microbe 2008; 4:337-49; Barnich et al., J. Clin. Invest. 2007; 117:1566-74; Barnich et al., J. Clin. Invest. 2007; 117: 1566-74. Thus, it is difficult to recapitulate the complexity of the gut environment in ex-vivo experiments using perhipheral blood cells. In addition, IL23R is exprssedon other cell types than Th17 cells [Awasthi et al., J. Immunol. 2009; 182: 5904-8], such as innate immune cells, which migh also play an important role in the context of CD. Thus, analyzing Th17 specific cytokines might not give us the complete picture about the protective role of this variant in CD and other associated disorders.

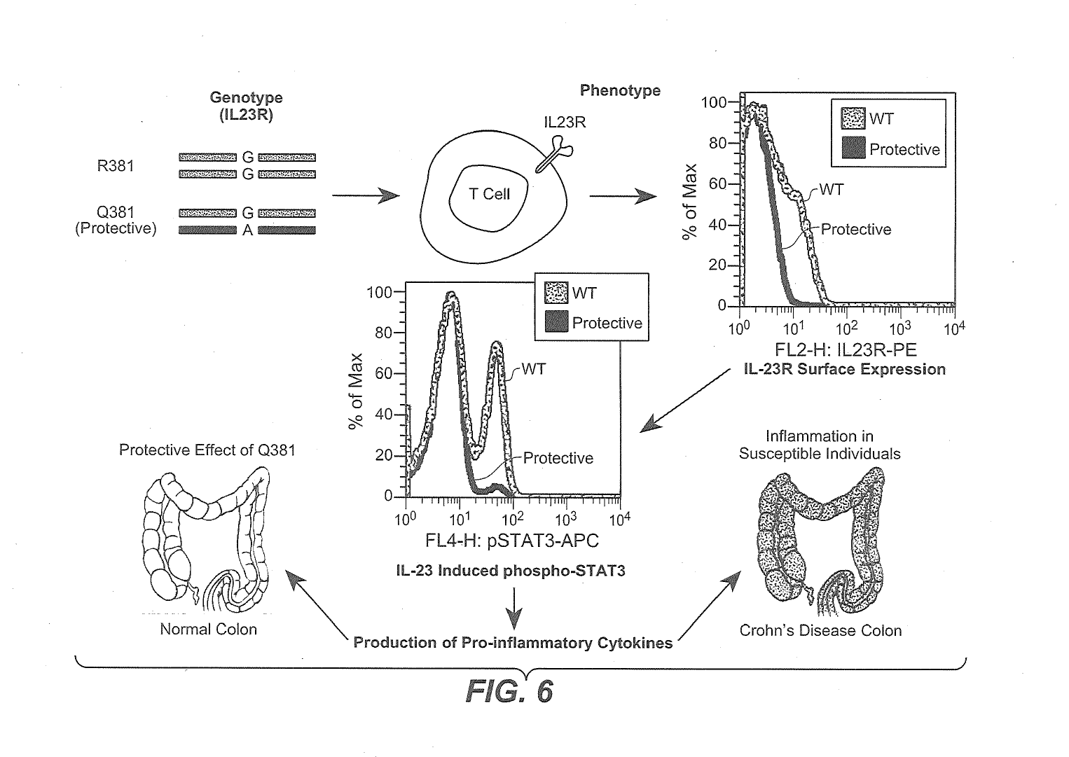

Our data indicate that IL-23R^{Q381} is a hypermorphic IL-23R allele that results in decreased population of IL-23 responsive cells leading to diminished IL-23 induced STAT3 phosphorylation. This provides an explanation for the protective role of R381Q in CD and other autoimmune disorders (FIG. 6) and further supports the hypothesis that blocking the IL-23 pathway may lead to improved therapeutics for autoimmune disorders like CD [Sandborn et al., Gastroenterology 2008; 135:1130-41] and psoriasis [Malefyt R., Expert Rev. Dermatol. 2008; 3: S13-D17].

### Methods

### Antibodies and ELISA

Information for all antibodies used in this study is summarized in supplemental table 1. A monoclonal antibody against human IL-23R (clone 20G3.4) was generated in mice, immunizing mice with a hIL-23R-Ig fusion protein. Unconjugated, biotinylated, and FITC labeled versions of this antibody were used in this study. IL-22 levels in serum of human healthy donors was measured using the IL-22 ELISA MAX Set Deluxe kit (BioLegend, Inc) according to the manufacturer's instructions. Human IL-17A levels were measured in supernatants from PBMCs isolated from genotype specific healthy donors and stimulated with anti-CD3 (2.5 μg/ml) and anti-CD28 (1 μg/ml antibodies +/−IL-23 (5 μg/ml) for 2 days. Supernatents were harvested and analyzed using hIL-17A ELISA (eBioscience) according to the manufactuer's instructions.

### IL-23R Constructs

Gateway recombination cloning technology (Invitrogen) was used to create all constructs. Full-length wild-type IL-23R coding sequence was PCR amplified using following primers:

forward 5′-3′
(SEQ. ID. NO. 1)
GGACAAGTTTGTACAAAAAAGCAGGCTTCACCATGAATCAGGTCACTATT
Reverse 5′-3′
(SEQ. ID. NO. 2)
GGGGACCACTTTGTACAAGAAAGCTGGGTCCTACTTTTCCAAGAGTGA

The full-length wild-type IL-23R coding sequence was PCR amplicied and the PCR PCR product (IL-23R^{R381}) was first cloned into pDONR²²¹ donor vector (Invitrogen, Carlsbad, Calif.). The R381Q variant was introduced into IL-23R^{R381} using a QuikChange XL site-directed mutagenesis kit (Stratagene) and verified by sequencing. pMSCVpuro retroviral expression vector (Clontech) was converted into a gateway compatible destination vector using the Gateway Vector Conversion System (Invitrogen). Constructs encoding IL-23R^{R381} and IL-23^{Q381} were subsequently transferred into a gateway adapted pMSCVpuro plasmid using LR Clonase, and final destination constructs were sequence verified.

### Genotyping

Blood samples were obtained from the Genentech blood donors program after written informed consent was provided. Ethical approval for the use of this material was obtained from the Western Institutional Review Board. Genomic DNA was isolated from 138 healthy Genentech donors. R381Q variant (rs11209026) was genotyped using Applied Biosystems TaqMan SNP genotyping assay (assay ID: C_{—}1272298_{—}10). Eighteen donors were identified as being IL-23R^{Q381} heterozygous (GA) and 1 homozygous (AA).

### BaF3 Cell Culture and Transduction

BaF3 cells (Palacios et al., Cell 1985; 41:727-34) were maintained in RPMI supplemented with 10% bovine calf serum, L-Glutamine and Penicillin-Streptomycin (Invitrogen, Carlsbad, Calif.). Conditioned medium from WEHI-3B cells was used as a source of IL-3 and added to the culture at 2% final concentration. A pMSCV-based plasmid encoding hIL-12R¹³¹ was introduced by electroporation, and positive single cell clones were identified and sorted by FACS into individual wells of 96-well plates. Human IL-23R^{R381} or IL-23R^{Q381} cDNA, cloned in the pMSCVpuro retroviral expression vector, was introduced into the same hIL-12R¹³¹ containing BaF3 subclones by standard retroviral transduction. 293T cells in combination with the retroviral packaging vector pCL-Eco (Imgenex) were used as a packaging system. Twenty-four hours after transduction, BaF3 cells were put in 1 μg/ml puromycin (Clontech) medium to select transduced cells. Single cell clones of IL-23R^{R381} and IL-23R^{Q381} with equal IL-23R surface expression levels were sorted inot individual wells of a 96-well plate by FACS, expanded and surface expression was verified by FACS. Three pairs of clones were identified with equal IL-23R surface expression. Relative IL-23R and IL12Rβ1 mRNA abundances in IL-23R^{Q381} and IL-23R^{Q381} clones were verified by qPCR.

### T-cell Lines

T cell lines were generated as previously described⁽²⁴⁻²⁶⁾. Briefly, CD4+ T cells were sorted from whole blood of IL-23R^{Q381} and IL-23R^{Q381} positive donors using a human whole blood CD4 selection kit (RoboSep; StemCell Technologies) according to the manufacturer's instructions (purity of the CD4+ cells after enrichment was >95-%). Cells were seeded at 5×10⁵ cells/ml and stimulated with a feeder mixture containing 1×106/ml irradiated (6,000 rad) allogenic PBMC and 1×105/ml irradiated (10,000 rad) JY cells, 1 μg/ml phytohemagglutinin (Sigma), and 200 IU/ml recombinant human IL-2 (Roche). Cells were cultured in Yssel's medium (Gemini Bio-Products) supplemented with 1% human serum. T cells were restimulated with feeder every 2 weeks. Cell surface expression of IL-23R was analyzed by flow cytometry 6 days after stimulation.

### T-cell Culture

CD4+ T cells were enriched from whole blood of IL-23R^{R381} and IL-23R^{Q381} positive donors using a Rosettesep Human CD4+ T Cell Enrichment kit (StemCell Technologies) according to the manufacturer's instructions. Cells were stimulated with anti-CD3 (2.5 μg/ml) and anti-CD28 (1 μg/ml) antibodies for 72 hours. IL-23R cell surface cell surface expression was analyzed by flow cytometry using biotinylated IL-23R antibody in combination with streptavidin-PE (eBioscience).

### Flow Cytometry

All data were collected on FacsCalibur and LSR II instruments (BD Biosciences) and analyzed using FlowJo software (Tree Star). A summary of all antibodies used in this study is provided below in Table 2, and antibodies were used according to the instructions of the manufacturer unless otherwise indicated.

TABLE 2

| Antigen | Manufacturer | Clone | Format | Application |
|---|---|---|---|---|
| IL23R | Genentech Inc | 20G3.4 | Biotin | Flow cytometry |
| IL23R | Genentech Inc | 20G3.4 | FITC | Flow cytometry |
| CCR6 | R&D Systems | FAB195A | APC | Flow cytometry |
| CXCR3 | BD Bioscience | 1C6/CXCR3 | PE-CY5 | Flow cytometry |
| | Pharmingen | | | |
| CD45RA | BD Bioscience | HI100 | FITC | Flow cytometry |
| | Pharmingen | | | |
| CD25 | BD Bioscience | M-A251 | FITC | Flow cytometry |
| | Pharmingen | | | |
| CCR4 | BD Bioscience | 1G1 | PECY7 | Flow cytometry |
| | Pharmingen | | | |
| CD45RO | BD Bioscience | UCHL1 | FITC | Flow cytometry |
| | Pharmingen | | | |
| CD4 | BD Bioscience | RPA-T4 | APC CY7 | Flow cytometry |
| | Pharmingen | | | |
| hIL-2 | R&D Systems | 5334 | pure | Neutralization |
| IL-22 | Genentech Inc | 3F11 | Alexa- | Flow cytometry |
| | | | Fluor 647 | |
| IFN-g | BD Bioscience | B27 | PECY7 | Flow cytometry |
| IL-17A | eBioscience | eBio64DEC17 | PE | Flow cytometry |
| IL-10 | eBioscience | JES3-9D7 | Pacific | Flow cytometry |
| | | | Blue | |
| pSTAT1 | BD Bioscience | 4a | Alexa- | Flow cytometry |
| (pY701) | Pharmingen | | Fluor 488 | |
| pSTAT3 | BD Bioscience | 4/P-STAT3 | Alexa- | Flow cytometry |
| (pY705) | Pharmingen | | Fluor 647 | |
| pSTAT5 | BD Bioscience | 47 | PE | Flow cytometry |
| (pY694) | Pharmingen | | | |
| CD3 | BD Bioscience | HIT3a | APC | Flow cytometry |
| | Pharmingen | | | |
| CD8 | BD Bioscience | RPA-T8 | APC | Flow cytometry |
| | Pharmingen | | | |
| IL12Rβ1 | R&D Systems | FAB839P | PE | Flow cytometry |
| CD28 | BD Bioscience | CD28.2 | pure | Stimulation |
| | Pharmingen | | | |
| CD3 | eBioscience | OKT3 | pure | Stimulation |
| CD28 | BD Bioscience | CD28.2 | pure | Stimulation |
| | Pharmingen | | | |
| CD3 | eBioscience | OKT3 | pure | Stimulation |
| CD161 | eBioscience | DX12 | PE-Cy5 | Flow cytometry |
| IL1R1 | R&D Systems | FAB269P | PE | Flow cytometry |

### IL-23R Cell Surface Expression

BaF3 cells were stained with biotinylated IL-23R antibody 20G3.4 in combination with streptavidin-PE (eBioscience). For intracellular staining we used FITC-conjugated anti IL-23R antibody. Isotype control antibodies were used as negative controls (BD biosciences).

### STAT Phosphorylation

Flow cytometric analyses of STAT phosphorylation was performed as previously described^{(26,27)}. Briefly, BaF3 cells were starved overnight in RPMI supplemented with 1% FBS. The next day cells were stimulated with 5 ng/ml of rhIL-23 (eBioscience) for 15 min at 37° C. Activation was blocked by immediate fixation in paraformaldehyde. A phospho-STAT3 specific antibody was used to detect activated STAT3.

T cells were starved overnight in Yssel's medium supplemented with monoclonal anti-human IL-2 antibody to neutralize IL-2. Cells were stimulated with 10 ng/ml of rhIL-23 or 10 ng/ml of rhIL-6 or 1000 IU/ml of IL-2 and incubated at 37° C. for 15 min. A phospho-STAT3, phospho-STAT1 and phospho-STAT5 specific antibodies were used to detect activated STAT3, STAT1 and STAT5.

Whole blood was stimulated with 10 ng/ml of IL-23 or 10 ng/ml of rhIL-6 and incubated at 37° C. for 15 minutes, fixed and lysed to halt signaling and lyse red blood cells. Cells were permeabilized and stained with phospho-STAT3 specific antibodies to detect activated STAT3.

### TH17 Cell Surface Staining on Freshly Isolated PBMC

Cells were stained as previously described⁽²⁸⁾. Briefly, CD4+ T cells were enriched from whole blood of IL-23R^{R381} and IL-23R^{Q381} positive donors using a Rosettesep Human CD4+ T Cell Enrichment kit (StemCell Technologies) according to the manufacturer's instructions. Cells were then labeled with anti-CCR6-APC, anti-CD25-FITC, anti-CD45RA-FITC, anti-CCR4-PECY7, anti-CXCR3-PECY5, anti-CD161-PE-Cy5 and anti-IL1R1-PE. TH17 cells were defined as CCR6+CCR4+ CXCR3-⁽²⁹⁾ In addition, CD4⁺CD45RO⁺CD 161⁺IL1R1⁺ cells were analyzed.

### Intracellular Cytokine Staining (ICS)

Intracellular staining was done as described.⁽²⁸⁾ Briefly, T cells were stimulated either with Dynabeads CD3/CD28 T Cell Expander (Invitrogen) (bead:cell ratio=1:10) or with 10 ng/ml PMA plus 500 ng/ml ionomycin. After 3 hrs, BD Golgi Plug with brefeldin A (BD Biosciences) was added to block secretion. After an additional 3 hrs, T cells were stained with green live/dead dye (Invitrogen). Cells were fixed in 3% paraformaldehyde and permeabilized with Perm/Wash buffer (BD Biosciences). Permeabilized T cells were stained with anti-hIFN-γ, anti-hIL-17, anti-hIL-10, anti-hIL-22 or isotype control antibodies (BD Biosciences).

Real-time quantitative RT-PCR

Total RNA was extracted from freshly isolated PBMC of IL-23R^{R381} and IL-23R^{Q381} positive donors using RNeasy Micro kit (Qiagen). High-Capacity cDNA Archive kit (Applied Biosystems) was used for cDNA synthesis. Transcripts were quantified by real-time quantitative PCR on an ABI PRISM 7900 sequence detector (Applied Biosystems) with Applied Biosystems predesigned TaqMan Gene Expression Assays. The following probes were used: RORC (Hs01076112) and IL-23R (Hs00332759). For each sample, mRNA abundance was normalized to the amount of GAPDH or RPL19 transcripts.

### Sequence Alignment by CLUSTAL W

IL-23R protein sequences from all the species available in Genbank were aligned with Clustal W in FASTA format (available from the website of the European Bioinformatics Institute.

### Statistical Analysis.

Statistical analysis was performed by unpaired student's t-test using Graph Pad Prism software with P<0.05 considered statistically significant.

### Example 1

### Reference List

1. Buonocore, S., Ahern, P. P., Uhlig, H. H., Ivanov, I I, Littman, D. R., Maloy, K. J., and Powrie, F. Innate lymphoid cells drive interleukin-23-dependent innate intestinal pathology. Nature 464:1371-1375.
2. Takatori, H., Kanno, Y., Watford, W. T., Tato, C. M., Weiss, G., Ivanov, I I, Littman, D. R., and O'Shea, J. J. 2009. Lymphoid tissue inducer-like cells are an innate source of IL-17 and IL-22. J Exp Med 206:35-41.
3. Peng, J., Yang, X. O., Chang, S. H., Yang, J., and Dong, C. IL-23 signaling enhances Th2 polarization and regulates allergic airway inflammation. Cell Res 20:62-71.
4. Langowski, J. L., Zhang, X., Wu, L., Mattson, J. D., Chen, T., Smith, K., Basham, B., McClanahan, T., Kastelein, R. A., and Oft, M. 2006. IL-23 promotes tumour incidence and growth. Nature 442:461-465.
5. Teng, M. W., Andrews, D. M., McLaughlin, N., von Scheidt, B., Ngiow, S. F., Moller, A., Hill, G. R., Iwakura, Y., Oft, M., and Smyth, M. J. IL-23 suppresses innate immune response independently of IL-17A during carcinogenesis and metastasis. Proc Natl Acad Sci USA 107:8328-8333.
6. Yamazaki, K., McGovern, D., Ragoussis, J., Paolucci, M., Butler, H., Jewell, D., Cardon, L., Takazoe, M., Tanaka, T., Ichimori, T., et al. 2005. Single nucleotide polymorphisms in TNFSF15 confer susceptibility to Crohn's disease. Hum Mol Genet. 14:3499-3506.
7. Rioux, J. D., Xavier, R. J., Taylor, K. D., Silverberg, M. S., Goyette, P., Huett, A., Green, T., Kuballa, P., Barmada, M. M., Datta, L. W., et al. 2007. Genome-wide association study identifies new susceptibility loci for Crohn disease and implicates autophagy in disease pathogenesis. Nat Genet. 39:596-604.
8. Hampe, J., Franke, A., Rosenstiel, P., Till, A., Teuber, M., Huse, K., Albrecht, M., Mayr, G., De La Vega, F. M., Briggs, J., et al. 2007. A genome-wide association scan of nonsynonymous SNPs identifies a susceptibility variant for Crohn disease in ATG16L1. Nat Genet. 39:207-211.
9. Libioulle, C., Louis, E., Hansoul, S., Sandor, C., Farnir, F., Franchimont, D., Vermeire, S., Dewit, O., de Vos, M., Dixon, A., et al. 2007. Novel Crohn disease locus identified by genome-wide association maps to a gene desert on 5p13.1 and modulates expression of PTGER4. PLoS Genet. 3:e58.
10. Franke, A., Hampe, J., Rosenstiel, P., Becker, C., Wagner, F., Hasler, R., Little, R. D., Huse, K., Ruether, A., Balschun, T., et al. 2007. Systematic association mapping identifies NELL1 as a novel IBD disease gene. PLoS One 2:e691.
11. 2007. Genome-wide association study of 14,000 cases of seven common diseases and 3,000 shared controls. Nature 447:661-678.
12. Parkes, M., Barrett, J. C., Prescott, N. J., Tremelling, M., Anderson, C. A., Fisher, S. A., Roberts, R. G., Nimmo, E. R., Cummings, F. R., Soars, D., et al. 2007. Sequence variants in the autophagy gene IRGM and multiple other replicating loci contribute to Crohn's disease susceptibility. Nat Genet. 39:830-832.
13. Duerr, R. H., Taylor, K. D., Brant, S. R., Rioux, J. D., Silverberg, M. S., Daly, M. J., Steinhart, A. H., Abraham, C., Regueiro, M., Griffiths, A., et al. 2006. A genome-wide association study identifies IL-23R as an inflammatory bowel disease gene. Science 314:1461-1463.
14. Raelson, J. V., Little, R. D., Ruether, A., Fournier, H., Paquin, B., Van Eerdewegh, P., Bradley, W. E., Croteau, P., Nguyen-Huu, Q., Segal, J., et al. 2007. Genome-wide association study for Crohn's disease in the Quebec Founder Population identifies multiple validated disease loci. Proc Natl Acad Sci USA 104:14747-14752.
15. Capon, F., Di Meglio, P., Szaub, J., Prescott, N. J., Dunster, C., Baumber, L., Timms, K., Gutin, A., Abkevic, V., Burden, A. D., et al. 2007. Sequence variants in the genes for the interleukin-23 receptor (IL-23R) and its ligand (IL12B) confer protection against psoriasis. Hum Genet. 122:201-206.
16. Rueda, B., Orozco, G., Raya, E., Fernandez-Sueiro, J. L., Mulero, J., Blanco, F. J., Vilches, C., Gonzalez-Gay, M. A., and Martin, J. 2008. The IL-23R Arg381Gln non-synonymous polymorphism confers susceptibility to ankylosing spondylitis. Ann Rheum Dis 67:1451-1454.
17. Parham, C., Chirica, M., Timans, J., Vaisberg, E., Travis, M., Cheung, J., Pflanz, S., Zhang, R., Singh, K. P., Vega, F., et al. 2002. A receptor for the heterodimeric cytokine IL-23 is composed of IL-12Rbeta1 and a novel cytokine receptor subunit, IL-23R. J Immunol 168:5699-5708.
18. Oppmann, B., Lesley, R., Blom, B., Timans, J. C., Xu, Y., Hunte, B., Vega, F., Yu, N., Wang, J., Singh, K., et al. 2000. Novel p19 protein engages IL-12p40 to form a cytokine, IL-23, with biological activities similar as well as distinct from IL-12. Immunity 13:715-725.
19. Harrington, L. E., Hatton, R. D., Mangan, P. R., Turner, H., Murphy, T. L., Murphy, K. M., and Weaver, C. T. 2005. Interleukin 17-producing CD4+ effector T cells develop via a lineage distinct from the T helper type 1 and 2 lineages. Nat Immunol 6:1123-1132.
20. Aggarwal, S., Ghilardi, N., Xie, M. H., de Sauvage, F. J., and Gurney, A. L. 2003. Interleukin-23 promotes a distinct CD4 T cell activation state characterized by the production of interleukin-17. J Biol Chem 278:1910-1914.
21. Lerch-Bader, M., Lundin, C., Kim, H., Nilsson, I., and von Heijne, G. 2008. Contribution of positively charged flanking residues to the insertion of transmembrane helices into the endoplasmic reticulum. Proc Natl Acad Sci USA 105:4127-4132.
22. Chaligne, R., Tonetti, C., Besancenot, R., Roy, L., Marty, C., Mossuz, P., Kiladjian, J. J., Socie, G., Bordessoule, D., Le Bousse-Kerdiles, M. C., et al. 2008. New mutations of MPL in primitive myelofibrosis: only the MPL W515 mutations promote a G1/S-phase transition. Leukemia 22:1557-1566.
23. Palacios, R., and Steinmetz, M. 1985. 11-3-dependent mouse clones that express B-220 surface antigen, contain Ig genes in germ-line configuration, and generate B lymphocytes in vivo. Cell 41:727-734.
24. Trifari, S., Sitia, G., Aiuti, A., Scaramuzza, S., Marangoni, F., Guidotti, L. G., Martino, S., Saracco, P., Notarangelo, L. D., Roncarolo, M. G., et al. 2006. Defective Th1 cytokine gene transcription in CD4+ and CD8+ T cells from Wiskott-Aldrich syndrome patients. J Immunol 177:7451-7461.
25. Yssel, H., and Spits, H.2002. Generation and maintenance of cloned human T cell lines. Curr Protoc Immunol Chapter 7:Unit 7 19.
26. Crellin, N. K., Garcia, R. V., and Levings, M. K. 2007. Flow cytometry-based methods for studying signaling in human CD4+ CD25+FOXP3+ T regulatory cells. J Immunol Methods 324:92-104.
27. Schulz, K. R., Danna, E. A., Krutzik, P. O., and Nolan, G. P. 2007. Single-cell phospho-protein analysis by flow cytometry. Curr Protoc Immunol Chapter 8:Unit 8 17.
28. Trifari, S., Kaplan, C. D., Tran, E. H., Crellin, N. K., and Spits, H. 2009. Identification of a human helper T cell population that has abundant production of interleukin 22 and is distinct from T(H)-17, T(H)1 and T(H)₂ cells. Nat Immunol 10:864-871.
29. Napolitani, G., Acosta-Rodriguez, E. V., Lanzavecchia, A., and Sallusto, F. 2009. Prostaglandin E2 enhances Th17 responses via modulation of IL-17 and IFN-gamma production by memory CD4+ T cells. Eur J Immunol 39:1301-1312.
30. Parham C., Chiricia M., Timans J., et al., A receptor for the heterodimeric cytokine IL-23 is composed of IL-12101 and a novel cytokine receptor subunit, IL-23R. J. Immunol. 2002; 168:5699-708.

1. A method of advising a treatment regimen to a patient having at least one symptom of an AID comprising:
(a) analyzing a tissue sample from said patient for an IL-23R LOF mutation, and
(b) advising said patient or their care provider on treatment options based on the presence or absence of said IL-23R LOF mutation,
wherein (i) the presence of the IL-23R LOF mutation results in the administration of an agent other than an IL-23 pathway antagonist, and (ii) the absence of the IL-23R LOF mutation results in the administration of an agent that may include an IL-23 pathway antagonist.

2. A method of treating a patient having at least one symptom of an AID comprising:
(a) analyzing a tissue sample from said patient for an IL-23R LOF mutation, and
(b) administering at least a therapeutically effective amount of a therapeutic based on the presence or absence of said IL-23R LOF mutation;
wherein the therapeutic administered, includes (i) an agent other than an IL-23 pathway antagonist when an IL-23R LOF mutation is present, and (ii) an agent that may include an IL-23 pathway antagonist if an IL-23R LOF mutation is not present.

3. The method of claim 2, wherein an IL-23 pathway antagonists is administered when an IL-23R LOF mutation is not found in the tissue sample.

4. The method of claim 2, wherein the patient has an AID.

5. The method of claim 4, wherein the AID is selected from the group consisting of:
ankylosing spondylitis, inflammatory bowel disease, dermatomyocitis and rheumatoid arthritis.

6. The method of claim 5, wherein the AID is IBD.

7. The method of claim 2, wherein the IL-23R LOF mutation results from the polymorphism R381Q.

8. The method of claim 7, wherein polymorphism results from the SNP rs11209026.

9. The method of claim 2 wherein IL-23R LOF mutation results from a SNP selected from the group consisting of: rs11209026, rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, and rs11209003.

10. The method of 2, wherein the other than IL-23 pathway antagonist is selected from the group consisting of: an aminosalicylate, a corticosteroid, an immunosuppressive agent, an antibody targeting other than an IL-23 pathway component or antigen binding fragment thereof, an antibiotic and anti-metabolic agent and a palliative therapy.

11. The method of claim 2, wherein the IL-23 pathway antagonist is directed against one or more IL-23 pathway components selected from the group consisting of: p40 (IL-12B), p19 (IL-23A), IL-12RB1, IL-23R, TYK2, JAK2, STAT-3.

12. In yet another embodiment, the invention provides a method of treating a patient having at least one symptom of chronic inflammation comprising:
(a) analyzing a tissue sample from said patient for an IL-23R LOF mutation, and
(b) administering at least a therapeutically effective amount of a therapeutic based on the presence or absence of said LOF mutation;
wherein the therapeutic administered, includes (i) an agent other than an IL-23 pathway antagonist when an IL-23R LOF mutation is present, and (ii) an agent that may include an IL-23 pathway antagonist if an IL-23R LOF mutation is not present.

13. The method of claim 12, wherein an IL-23 pathway antagonists is administered when an IL-23R LOF mutation is not found in the tissue sample.

14. The method of claim 12, wherein the patient has an AID.

15. The method of claim 14, wherein the AID is selected from the group consisting of: ankylosing spondylitis, inflammatory bowel disease, dermatomyocitis and rheumatoid arthritis.

16. The method of claim 15, wherein the AID is IBD.

17. The method of claim 12, wherein the IL-23R LOF mutation results in the polymorphism R381Q.

18. The method of claim 17, wherein the polymorphism results from the SNP rs11209026.

19. The method of claim 12, wherein IL-23R LOF mutation is selected from the group consisting of the SNPs: rs11209026, rs1884444, rs11465779, rs11465797, rs7530511, rs41313262, rs10789230, rs6669582, rs12567232, rs9988642, rs10889677, rs10889676, rs1343151, rs11209026, rs11465804, rs2201841, rs11465802, rs2902440, rs1004819, rs2064689, rs11209008, and rs11209003.

20. The method of claim 12, wherein the other than IL-23 pathway antagonist is selected from the group consisting of: an aminosalicylate, a corticosteroid, an immunosuppressive agent, an antibody targeting other than an IL-23 pathway component or antigen binding fragment thereof, an antibiotic and anti-metabolic agent and a palliative therapy.

21. The method of claim 12, wherein the IL-23 pathway antagonist is directed against one or more IL-23 pathway components selected from the group consisting of: p40 (IL-12B), p19 (IL-23A), IL-12RB1, IL-23R, TYK2, JAK2, STAT-3.

22. The method of claim 16, wherein the tissue sample is derived from a colonic tissue biopsy.

23. The method of claim 22, wherein the colonic tissue is selected from the group consisting of terminal ileum, the ascending colon, the descending colon, and the sigmoid colon.

24. The method of claim 16, wherein the colonic tissue is from an inflamed colonic area or from a non-inflamed colonic area.

25. The method of claim 24, wherein the colonic tissue is acutely inflamed.

26. The method of claim 24, wherein the colonic tissue is chronically inflamed.

27. A method of assessing the function of an IL-23 responsive cell, comprising: (a) isolating the cell, (b) detecting an IL-23R loss-of-function mutant in said cell, and (b) wherein the presence of IL-23R LOF mutant correlates to diminished cell function.

28. The method of claim 27, wherein the diminished function is Th17-induced inflammation.

29. The method of claim 27, wherein the diminished function is surface expression of IL-23R.

30. The method of claim 27, wherein the diminished function is a reduced Th17 cytokine response profile.

31. The method of claim 27, wherein the diminished function is reduced STAT3 phosphorylation.

32. The method of claim 27, wherein the diminished function is reduced expression of RORyt.

33. The method of claim 27, wherein the diminished function is reduced expression of GATA-3.

34. The method of claim 27, wherein the diminished function is reduced expression of matrix metalloprotease MMP9.

35. The method of claim 27, wherein the IL-23 responsive cell is selected from the group consisting of: dendritic cells, T cells, including αβ and γδ T cells, NK cells, including NKL, monocytes, macrophages, B cells αβ and γδ T cells as well as innate leukocytes.

36. The method of claim 33, wherein the IL-23 responsive cell is a T cell.

37. (canceled)


SEQUENCE LISTING (2 sequences)

SEQ ID NO: 1 - DNA, 50 bp, Artificial sequence
  sequence is synthesized
ggacaagtttgtacaaaaaagcaggcttcaccatgaatcaggtcactatt

SEQ ID NO: 2 - DNA, 48 bp, Artificial sequence
  sequence is synthesized
ggggaccactttgtacaagaaagctgggtcctacttttccaagagtga